(19) United States
(12) Patent Application Publication
(10) Pub. No.: US 2014/0288427 A1
(43) Pub. Date: Sep. 25, 2014

(54) DEVICES AND METHODS FOR TISSUE NAVIGATION

(71) Applicant: James K. Wall, San Francisco, CA (US)

(72) Inventor: James K. Wall, San Francisco, CA (US)

(21) Appl. No.: 14/283,164

(22) Filed: May 20, 2014

Related U.S. Application Data
(63) Continuation-in-part of application No. 12/753,492, filed on Apr. 2, 2010.
(60) Provisional application No. 61/166,421, filed on Apr. 3, 2009.

(51) Int. Cl. A61B 1/313; A61B 17/32; A61B 19/00; A61B 8/08

(52) U.S. Cl. CPC A61B 1/3135; A61B 8/0841; A61B 17/320016; A61B 19/5244
USPC 600/439

(57) ABSTRACT

A device for use in a process that involves accessing an epidural space includes: an elongated member having a distal end and a proximal end; an ultrasound component located at the distal end of the elongated member; a rigid tubular member having a tapered distal end, a proximal end, and a lumen extending between the distal end and the proximal end; wherein the ultrasound component is configured to emit ultrasound in a distal direction, and to receive reflected ultrasound; and wherein the elongated member is slidably disposed in the lumen of the tubular member.

### RELATED APPLICATION DATA

This application is a continuation-in-part of U.S. patent application Ser. No. 12/753,492, filed on Apr. 2, 2010, pending, which claims priority to and the benefit of U.S. Provisional Patent Application No. 61/166,421, filed on Apr. 3, 2009, expired, the entire disclosures of both of which are expressly incorporated by reference herein.

### GOVERNMENT'S RIGHTS

This invention was made with Government support under Grant No. 0848916 awarded by the National Science Foundation. The Government has certain rights in this invention.

### FIELD

The field of the application relates to devices and methods for accessing a space in a body, and more particularly, for devices and methods for accessing epidural space.

### BACKGROUND

Epidural anesthesia blocks pain sensation at nerve roots that branch directly from the spinal cord by bathing them with local anesthetic agents or opioids delivered to the epidural space, a small region adjacent to the outer protective covering of the spinal cord. This route of drug delivery provides an effective method for pain control during childbirth, major surgery, and chronic back pain as well as many other types of pain that may be debilitating in nature or affect a patient's quality of life.

Accessing the epidural space to administer anesthetic remains challenging due to its small size and proximity to the spinal cord and risk of complications such as infections and debilitation. Furthermore, the currently accepted method of blindly accessing the epidural space with a straight needle is often a time consuming process of trial and error that carries a complication rate of 2-20%. The excessive time demands of placement and threat of complications result in hesitation and underutilization of epidural anesthesia. The trial and error process of epidural anesthesia development frequently is associated with uncertainties in positioning of the anesthetic delivery device within the body.

The healthcare practitioner is frequently challenged with identifying the proper trajectory to insert the device to avoid hitting bony structures that often requires device retraction and re-engagement. Specifically, knowledge of engagement of the ligamentum flavum represents an important step in advancing into the epidural space in a safe and controlled manner since this must be traversed to complete the procedure. Currently, these methods are done blindly by inserting a sharp needle and either feeling the device hit bone or by a loss-of-resistance technique that is the standard practice for epidural space detection. This mechanism of detection is at times inaccurate due to the state of the ligamentum flavum, and can lead to inadvertent puncture of the dura or damage to the tissue in the area of the spinal column as well as failure of pain control. These current methods that require significant training to proficiency and detection are subjective in nature.

One of the significant barriers to ease of use for epidural anesthesia is the ability to accurately determine a path to the ligamentum flavum. The existing technique of blindly inserting devices into the back often leads to a trial and error process of advancing the device toward to the ligamentum flavum and then to the epidural space.

Finding the ligamentum flavum includes the ability to avoid hitting the bone lining the passage from the skin to the epidural space. Current techniques typically correct for bone impingement by partially retracting the device, reorienting the device, and then advancing the device again. This technique can be a process of trial and error, leading to pain and tissue damage for the patient as well as delaying epidural space access and anesthetic delivery.

For the foregoing reasons, Applicants of the subject application determine that it would be desirable to have a device that assists a doctor in determining a desired entry location and/or a desired entry angle for a device to access the epidural space. It would also be desirable to have a device that detects the presence of bony or dense structures surrounding the epidural space accessing device, or lack thereof, during advancement toward the ligamentum flavum and epidural space. Disclosed herein are embodiments of a device and method that assist a user in determining a desired entry location and/or a desired entry angle for a device to access the epidural space prior to puncturing a patient's skin for introduction of the device. Also, disclosed herein are embodiments of a device and method that assist a user in determining a desired entry location and/or a desired entry angle for a device to access the epidural space after puncturing a patient's skin and during advancement of the device.

### SUMMARY

In accordance with some embodiments, a device for use in a process that involves accessing an epidural space includes: an elongated member having a distal end and a proximal end; an ultrasound component located at the distal end of the elongated member; a rigid tubular member having a tapered distal end, a proximal end, and a lumen extending between the distal end and the proximal end; wherein the ultrasound component is configured to emit ultrasound in a distal direction, and to receive reflected ultrasound; and wherein the elongated member is slidably disposed in the lumen of the tubular member.

In accordance with other embodiments, a device for use in a process that involves accessing an epidural space includes: an elongated member having a distal end and a proximal end; an ultrasound component located at the distal end of the elongated member, wherein the ultrasound component is configured to receive reflected ultrasound, and generate an output based on the reflected ultrasound; a rigid tubular member having a tapered distal end, a proximal end, and a lumen extending between the distal end and the proximal end, wherein the elongated member is slidably disposed in the lumen of the tubular member; and a processing unit communicatively coupled to the ultrasound component, wherein the processing unit is configured to generate signals based on the output from the ultrasound component, the signals representing information for presentation in a display, wherein the information comprises a series of lines, each of the lines corresponding to a respective tissue depth.

In accordance with other embodiments, a device for use in a process that involves accessing an epidural space includes: an ultrasound component having a distal end, a proximal end, and a body extending between the distal end and the proximal end, the distal end having an end surface facing in a distal direction, the body having a circumferential surface; a first channel at the circumferential surface; and a second channel at the end surface.

Other and further aspects and features will be evident from reading the following detailed description of the embodiments, which are intended to illustrate, not limit, the invention.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates an epidural space accessing device in accordance with some embodiments.

FIG. 2 illustrates an implementation of the device of FIG. 1, particularly showing the device having an audio device in accordance with some embodiments.

FIGS. 3A-3D illustrate a method of accessing an epidural space in accordance with some embodiments.

FIG. 4 illustrates another device for accessing an epidural space in accordance with other embodiments.

FIG. 5 illustrates another device for accessing an epidural space in accordance with some embodiments.

FIG. 6 illustrates another epidural space accessing device, particularly showing the device having a plurality of light sources in accordance with some embodiments.

FIG. 7 illustrates another epidural space accessing device, particularly showing the device having a display panel in accordance with some embodiments.

FIG. 8 illustrates another epidural space accessing device in accordance with other embodiments, particularly showing the epidural space accessing device using an optical technique.

FIG. 9 illustrates another epidural space accessing device having an ultrasound transducer in accordance with some embodiments.

FIG. 10 illustrates an external device that uses ultrasound along a path similar to the path that the epidural space accessing device will take, to assess different structures between the skin and the epidural space to enable proper positioning and entry of an epidural space accessing device.

FIG. 11 illustrates an external device that uses ultrasound from an angle generally perpendicular to the approximate path that the epidural space accessing device will take, to assess different structures between the skin and the epidural space to enable proper positioning and entry of an epidural space accessing device.

FIG. 12 illustrates a device having a transmitter in accordance with some embodiments.

FIG. 13 illustrates another epidural space accessing device in accordance with other embodiments.

FIG. 14 illustrates parts of the epidural space accessing device of FIG. 13 in accordance with some embodiments.

FIG. 15 illustrates a transducer component in accordance with some embodiments.

FIG. 16 illustrates a graphical user interface in accordance with some embodiments.

FIG. 17 illustrates another graphical user interface in accordance with other embodiments.

FIGS. 18A-18G illustrate a method of using the epidural space accessing device of FIG. 13 in accordance with some embodiments.

FIGS. 19A-19B illustrate examples of line patterns that may be displayed in a screen of a user interface.

FIGS. 20A-20B illustrate an embodiment of a device implemented using a handheld communication device.

### DETAILED DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

In one aspect, embodiments described herein are directed to an epidural space accessing device, and method of using such device. As used in this specification, the term “epidural space accessing device” may refer to a device that is used to insert (e.g., through an incision or puncture hole) through a patient's skin in an epidural procedure, or it may refer to a device that is externally used outside the patient that assists the doctor in an epidural procedure. In some embodiments, the epidural space accessing device is shaped in an elongated needle-like form and possesses a means for propagating and/or detecting waves created from ultrasound-based diagnostic medical imaging technology or even through the oscillation of a syringe plunger. In certain configurations the epidural space accessing device does not include a detecting element, and a separate detecting device is used to determine the location of the epidural space accessing device in the epidural space.

Other embodiments of the epidural space accessing device include one or more light sources for emitting light and one or more sensors for sensing light reflected from surrounding tissue in the region of the epidural space. The light may be in the visible and/or non-visible spectrum. Using the reflected light, the device can sense the opacity of surrounding tissues in the region of the epidural space. The light source(s) and the light sensor(s) for emitting and sensing light may either be integral components of the space accessing device or separate components that are used in conjunction with the space accessing device.

Other embodiments of the epidural space accessing device include components for creating and detecting an electric or magnetic field around spinal processes. One such embodiment utilizes electrical impedance tomography to assess the location of the device sensor element relative to the surrounding tissue.

In another aspect, embodiments of the devices and methods are for detecting a line of sight path for epidural access devices. Such methods use traditional echography transducers and elastography to determine the proper angular orientation for the device. In some embodiments, these devices and methods involve using an echography transducer (such as an “A” mode echography) placed at a location near estimated point of device insertion, and at an orientation in-line with the approximate line of travel of an epidural space accessing device. Through imaging and feedback, the proper entry location and/or angular orientation of the device is achieved. Other embodiments use a mechanism that transmits low frequency sound waves toward the epidural space which are then measured to determine the harmonics reflected back. These harmonics allow the user to identify the location of fluids in and around the spinal column. Still other embodiments of these methods include stimulating the muscles surrounding the spinal processes with a contact probe having one or more electrodes, and then sensing the stimulation using computerized devices to visualize bony, non-bony and less dense tissue.

In yet another aspect, embodiments described herein relate to methods of detecting the presence or absence of bony structures near an epidural space accessing device before and/or when the device is placed in a patient's body. Some of these methods function by detecting the magnitude of sound waves propagating through bone or dense structures in order to assess their proximity. Other methods proceed by emitting light laterally and/or longitudinally from the surface of the epidural space accessing device. The resultant opacity of surrounding tissue is measured allowing one to calculate the proximity of the epidural space accessing device to bony or dense structures. Similar methods operate by detecting differences in the mechanical impedance of soft tissue and bone. In some embodiments, such methods include oscillating a syringe plunger to create low frequency pressure waves and then detecting the low frequency pressure waves. Variations of this method relate to creating a resonant tone that is disrupted allowing a computerized or electronic device to determine the location of a spinal process through a change in tone. Still other similar methods relate to creating an electric field around spinal processes and measuring the electrical impedance to determine the presence or absence of the bony structures surrounding the epidural space accessing device.

The various embodiments will now be described in more detail with reference to the figures.

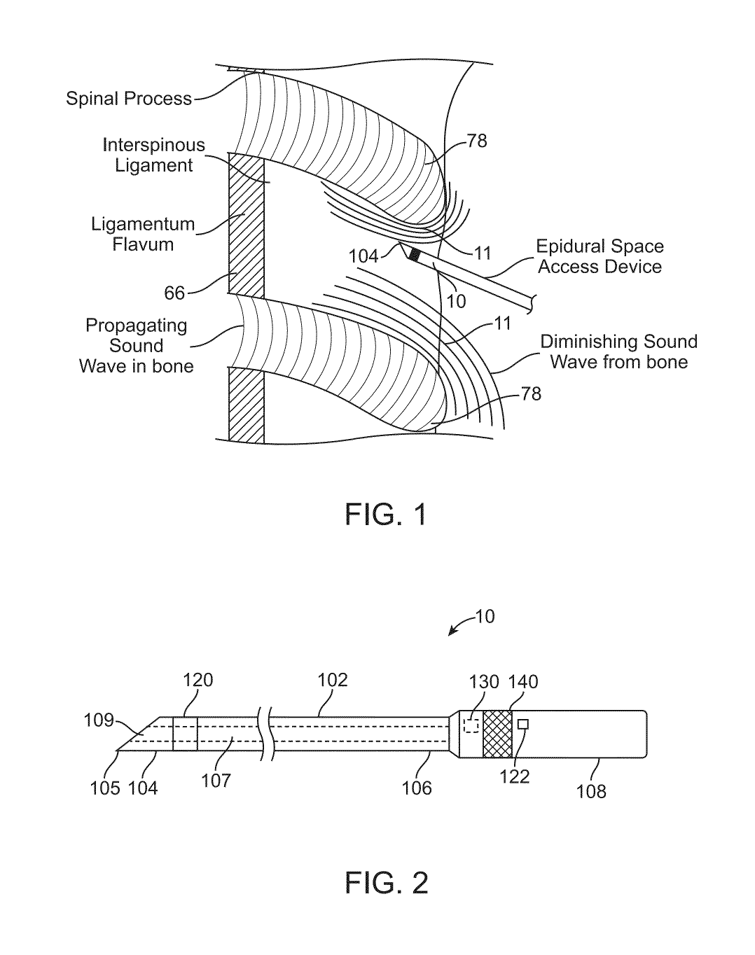

In some embodiments represented by FIG. 1, the position of the epidural space accessing device 10 in a patient's body relative to bony or dense structures, such as spinal process 78 is detected using sound waves 11. These waves are propagated from the epidural space accessing device 10 to bones 78 or dense structures proximate to the epidural space accessing device 10 entry location and trajectory. These waves are typically created using piezoelectric crystals, ceramics and other ultrasound transducers known to those of ordinary skill in the art. Examples of these materials include: berlinite, quartz, Rochelle salt, topaz, tourmaline-group minerals, gallium orthophosphate, langasite, barium titanate, lead titanate, lead zirconate titanate, potassium niobate, lithium niobate, lithium tantalite, sodium tungstate, Ba₂NaNb₅O₅, Pb₂KNb₅O₁₅, polyvinylidine fluoride, sodium potassium niobate and bismuth ferrite.

Determining the position of the epidural space accessing device 10 is accomplished by detecting the magnitude of the sound waves propagated. The bones 78 or dense structures act as acoustic sound boards that enable the user to determine the location of a portion of the epidural space accessing device 10 in the body. In performing this function, the portion of the epidural space accessing device 10 inserted may be the distal end 104 of the epidural space accessing device 10 or some component near the distal end. However, embodiments described herein are not limited to insertion of any particular length or amount of the epidural space accessing device 10.

FIG. 2 illustrates an epidural space accessing device 10 in accordance with some embodiments, particularly showing one implementation of the device 10 of FIG. 1. The device 10 includes a shaft 102 having a distal end 104 and a proximal end 106, and a handle 108 coupled to the proximal end 106 of the shaft 102. The distal end 104 has a tip 105 for cutting (e.g., puncturing) tissue. The shaft 102 also has a lumen 107 extending between the distal end 104 and the proximal end 106, and a distal opening 109 that is in fluid communication with the lumen 107. The lumen 107 is for housing another device (such as a tissue cutting device, a catheter, etc.) and/or for delivering fluid into a body through the distal opening 109. The device 10 also includes a transducer 120 coupled to the distal end 104 of the shaft 102, and a control 122 located at the handle 108 for operating (e.g., activating) the transducer 120 during use. The control may be a button, a sliding mechanism, or other actuating device. The transducer 120 is configured to emit acoustic signals and to sense reflected acoustic signals during use. The device 10 also includes a processor 130 communicatively coupled to the transducer 120, and an indicator 140. The indicator 140 is configured to provide a sensory indication for assisting a user to identify a desired entry path to access an epidural space. In other embodiments, instead of having one transducer 120 for transmitting and sensing acoustic signals, the device 10 may include a first transducer dedicated for transmitting acoustic signals, and a second transducer dedicated for sensing acoustic signals.

In the illustrated embodiments, the indicator 140 is implemented using an audio device, such as a speaker, which provides audio signal to the user during use. The audio device 140 is located at the handle 108. In other embodiments, the audio device 140 may be located at the shaft 102. In further embodiments, the audio device 140 may be communicatively coupled to the shaft 102 or the handle 108 via a cable or a wireless transmitter.

In some embodiments, the audio device 140 is configured to provide the audio signal when the distal end 104 of the shaft is at a desired entry location for accessing the epidural space. In other embodiments, the audio device 140 is configured to provide the audio signal when the distal end 104 of the shaft is at a desired entry angle for accessing the epidural space. In further embodiments, the audio device 140 is configured to provide the audio signal when the distal end 104 of the shaft is both at a desired entry location and at a desired entry angle for accessing the epidural space.

In other embodiments, the audio device 140 is configured to provide the audio signal when the distal end 104 of the shaft is not at a desired entry location for accessing the epidural space. In other embodiments, the audio device 140 is configured to provide the audio signal when the distal end 104 of the shaft is not at a desired entry angle for accessing the epidural space. In further embodiments, the audio device 140 is configured to provide the audio signal when the distal end 104 of the shaft is both not at a desired entry location and not at a desired entry angle for accessing the epidural space.

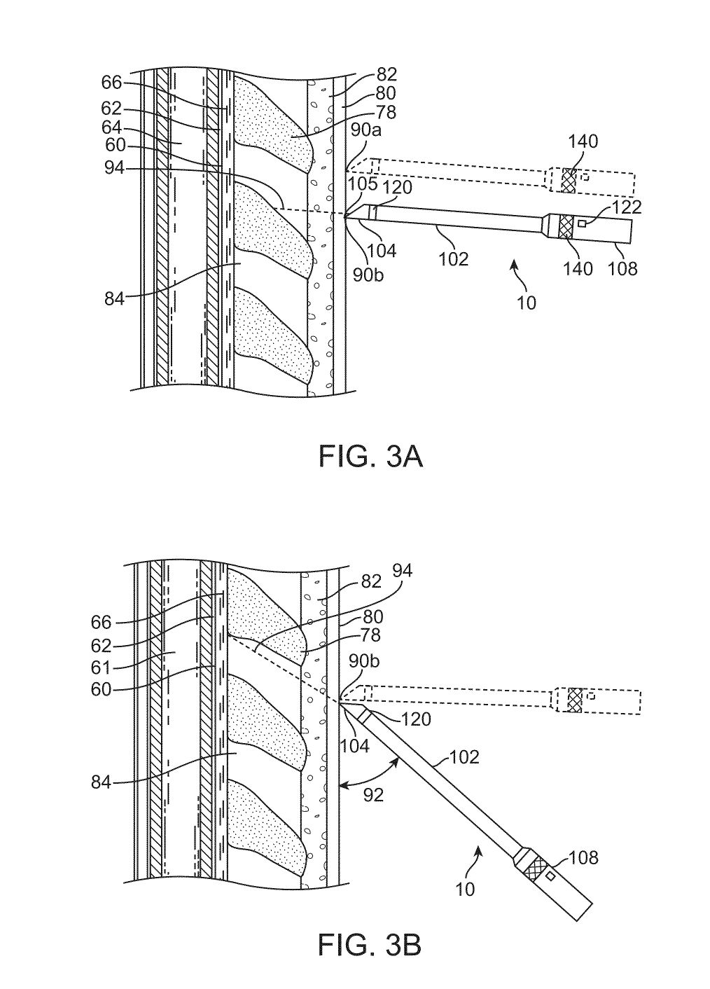

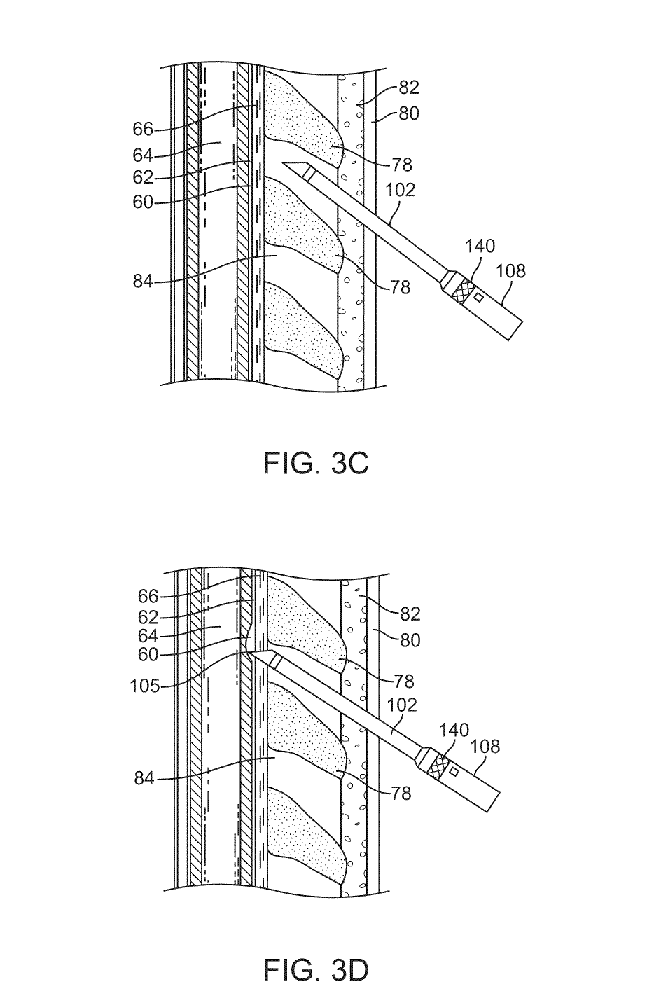

FIGS. 3A-3D illustrate a method of using the device 10 of FIG. 2 to access the epidural space in accordance with some embodiments. The epidural space is a potential space that is initially in a collapsed configuration, and is enlarged when the tissues that bound it are separated. As shown in FIG. 3A, the tissues that define the epidural space 60 including the dura mater (or dura 62) which is a protective covering that sheaths the spinal cord 64, the ligamentum flavum 66 which is a ligament adjacent to the dura 62 that runs longitudinally along the spinal column, and the bony sides of the vertebral canal. To access the epidural space 60, the patient is positioned either seated or on his/her side, and is instructed to flex the back outward to maximize the spacing between the outer vertebral components. The spinal processes 78 are palpated, and the interlaminar space is estimated. In order to reach the epidural space 60, the distal end 104 of the shaft 102 will need to penetrate the skin 80, soft tissue 82, and interspinous ligament 84.

Before the device 10 is used to penetrate the skin 80, the doctor (user) may use the device 10 to determine an entry point and an entry orientation (which collectively define an entry trajectory/path) at the patient's skin 80. In particular, the doctor may place the device 10 at a location that the doctor believes is close to a desired entry point. For example, the doctor may place the distal end 104 at location 90a (as represented by the dashed object in FIG. 3A). The transducer 120 is then activated using the control 122 to emit an acoustic signal towards an interior region in the body. In some cases, to provide or improve the acoustic coupling between the transducer 120 and the patient's skin 80, the transducer 120 may be located at the tip of the device 10, and/or the patient's skin 80 may be applied with a layer of gel. Alternatively, the distal tip 105 of the device 10 may be inserted slightly into the patient's skin 80 (e.g., placed within the skin layer 80 or within the tissue layer 82) to allow the transducer 120 to acoustically couple to the patient's skin. The transducer 120 is also used to sense signal reflected back from within the interior region in the body. In the example shown, because the distal end 104 at position 90a is close to the spinal process 78, the acoustic signal transmitted from the transducer 120 will be reflected back from the spinal process 78, and be sensed by the transducer 120. The processor 130 is configured to determine the time it takes for a transmitted signal to be reflected back to the transducer 120. In the example, because of the close proximity between the spinal process 78 and the transducer 120, the time it takes for the signal to be transmitted and reflected back will be relatively short. Also, the reflected signal may have an amplitude that is relatively strong because the reflected signal is not significantly attenuated by much soft tissue before it reaches back to the transducer 120 (or another sensing mechanism). As such, using the timing information and/or the amplitude information, the processor 130 may determine that the current position 90a of the distal end 104 is close to bone 78, and is therefore not desirable for accessing the epidural space. Accordingly, the processor 130 does not activate the indicator 140 to emit a signal to indicate that a desirable entry location has been reached. Alternatively, the processor 130 may cause the indicator 140 to emit a signal to let the doctor know that a desired entry position has not been reached.

Upon knowing that a desired entry trajectory has not been reached, the doctor continuously repositions the device 10. When the distal end 104 is at position 90b, the transducer 120 is further away from the bony structures. The entry position 90b for the distal end 104 is better than the entry position 90b, because at entry position 90b, the distal end 104 may be advanced further into the patient without hitting bone 78. However, the device 10 may still not be in an ideal position for accessing the epidural space 60 because the orientation of the device 10 is such that advancement of the device 10 along trajectory 94 will eventually result in the device 10 hitting the spinal process 78. As such, the processor 130 still does not activate the indicator 140 to emit a signal to indicate that a desirable entry trajectory has been reached. Alternatively, the processor 130 may cause the indicator 140 to emit a signal to let the doctor know that a desired entry trajectory has not been reached.

Upon knowing that a desired entry trajectory still has not been reached, the doctor continuously repositions the device 10, e.g., by translating, tilting, or both translating and tilting, the device 10 using the handle 108. When the device 10 is positioned like that shown in FIG. 3B, the trajectory 94 (as defined by the entry point 90b and the entry angle 92 relative to the patient's skin 80) will go through the interspinous ligament 84 between the spinal processes 78 to reach the ligamentum flavum 66. At this position of the device 10, the processor 130 determines that a desired entry trajectory has been obtained. Accordingly, the processor 130 then activates the indicator 140 to emit a signal to indicate to the doctor that a desired entry trajectory has been reached. Alternatively, the processor 130 may stop the indicator 140 from emitting a signal that indicates that a desired entry trajectory has not been reached.

In some embodiments, the processor 130 may determine that a desired entry trajectory has been reached using timing information (e.g., time it takes for an acoustic signal to reflect back to the transducer 120) and/or amplitude information (e.g., amplitude of reflected signal). In the configuration shown in FIG. 3B, the time it takes for an acoustic signal to reflect back from a bony structure to the transducer 120 is relatively longer (longer than the configurations shown in FIG. 3A), and the reflected acoustic signal strength is relatively low (compared to the configurations shown in FIG. 3A).

Upon knowing, through the “successful” signal (or lack of “unsuccessful” signal) provided by the device 10, that the desired entry trajectory for accessing the epidural space 60 has been achieved, the doctor then advances the device 10 along the trajectory 94 to puncture the skin 80, then the tissue 82, and then the interspinous ligament 84 (FIG. 3C). As shown in the illustrated embodiments, because the desired entry trajectory 94 has been established using the device 10 before the device 10 punctures the patient's skin 80 (or before the distal tip of the device passes through the tissue 82), the device 10 does not hit any bony structure as the device 10 is advanced towards the epidural space 60.

In some embodiments, after the device 10 has penetrated the patient's skin 80, the transducer 120 continues to emit acoustic signals and receive reflected acoustic signals. Also, the processor 130 continues to process the reflected acoustic signals to determine whether the device 10 is being desirably advanced through the tissue 82 and the interspinous ligament 84. In some cases, if the processor 130 determines that the device 10 is too close to the spinal process 78, the processor 130 then generates a signal to activate the indicator 140, thereby informing the doctor that the trajectory of the device 10 needs to be adjusted. In some embodiments, the indicator 140 may also inform the user how to adjust the device 10 (e.g., by informing the user the direction of adjustment).

As shown in FIG. 3D, the doctor continues to advance the device 10 until the distal tip 105 has reached the epidural space 60. Various techniques may be employed to determine whether the distal tip 105 has reached the epidural space 60. In some embodiments, the proximal end of the handle 108 is coupled to a fluid source (e.g., a syringe). As the doctor is advancing the device 10, the doctor uses the syringe to apply fluid delivery pressure through the lumen 107 of the shaft 102. As the distal end 104 goes through the skin 80, tissue 82, the interspinous ligament 84, and ligamentum flavum 66, the distal opening at the distal end 104 is blocked by these tissues. Therefore, the doctor will sense high pressure at the syringe as the doctor advances the distal end 104 through these tissues. As soon as the distal tip 105 has gone past the ligamentum flavum 66, the distal tip 105 is in the epidural space 60, and the opening 109 at the distal end 104 is no longer blocked by tissue. As such, the doctor will sense that the pressure being applied at the syringe has dropped, thereby knowing that the distal tip 105 has reached the epidural space 60.

Once the distal tip 105 is placed in the epidural space 60, the doctor then uncouples the syringe from the handle 108, and inserts a catheter (not shown) through the handle into the lumen 107 of the shaft 102. The catheter is advanced until it exits through the distal opening 109 at the distal end 104 of the shaft 102, and is desirably placed in the epidural space 60. The catheter may then be used to deliver fluid, such as saline, pain medication, or other substance into the epidural space 60. Alternatively, the shaft 102 itself may be used to deliver fluid into the epidural space 60. For example, the handle 108 may be directly coupled to a fluid source, such as a source of saline, pain medication, or any other drugs, and the lumen 107 of the shaft 102 is then directly used to deliver such fluid into the epidural space 60. In other embodiments, the device 10 may function as an introducer sheath that allows other devices, such as a tissue cutter, to be delivered to the epidural space 60 therethrough.

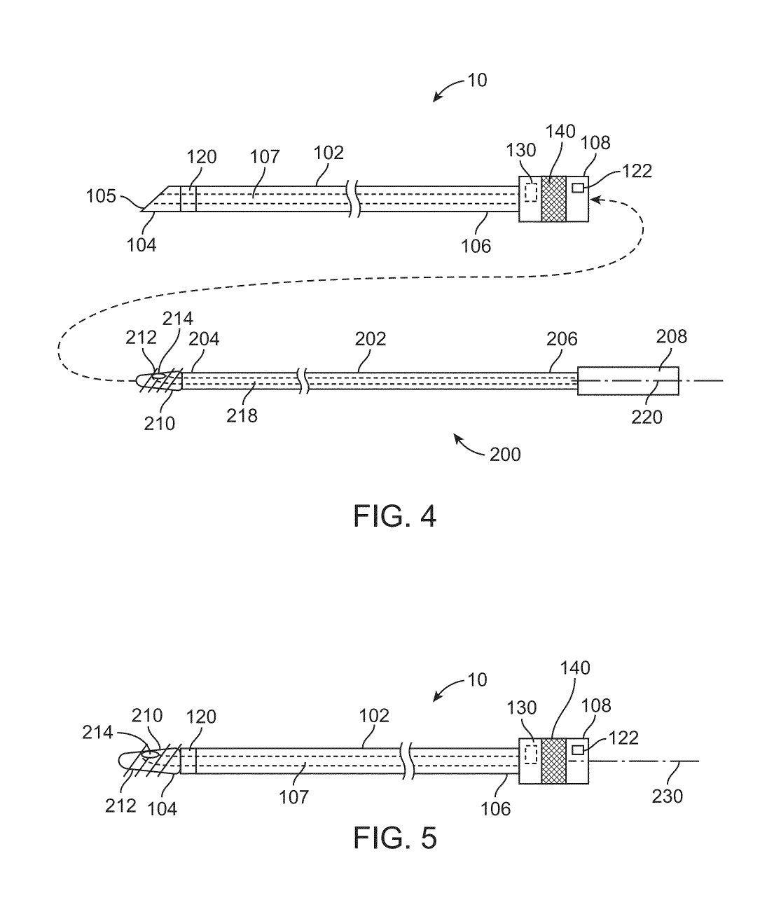

Also, in other embodiments, the distal tip 105 of the shaft 102 may not need to penetrate through the ligamentum flavum 66. In such cases, when the distal tip 105 reaches the ligamentum flavum 66, another device may be placed through the lumen 107 of the shaft 102, and may be deployed out of the distal opening 109 at the distal end 104 of the shaft 102 for penetrating the ligamentum flavum 66. FIG. 4 illustrates a device 200 that may be used to penetrate through the ligamentum flavum 66. In some embodiments, the device 200 may be considered to be a part of the device 10. The device 200 includes a shaft 202 with a distal end 204 and a proximal end 206, and a handle 208 coupled to the proximal end 206. The device 200 also includes a tissue-cutting portion 210 at the distal end 204. As shown in the illustrated embodiments, the tissue-cutting portion 210 includes threads 212 for cutting and engaging tissue at the ligamentum flavum 66, and an opening 214. The device 200 also includes a lumen 218 extending between the distal end 204 and the proximal end 206, and is in fluid communication with the distal opening 214. During use, after the distal end 104 of the shaft 102 is placed next to the ligamentum flavum 66, the device 200 is then inserted into the lumen 107 through the handle 108. The tissue-cutting portion 210 is then deployed out of the distal opening at the distal end 104 of the device 10, and is used to engage the ligamentum flavum 66. In particular, the doctor may rotate the handle 208 about the longitudinal axis 220 to screw the tissue-cutting portion 210 into the ligamentum flavum 66. The doctor continues to advance the tissue-cutting portion 210 by screwing action until the opening 214 at the tissue-cutting portion 210 has passed through the ligamentum flavum 66 into the epidural space 60. Using the tissue-cutting portion 210 with the screw threads 212 to penetrate the ligamentum flavum 66 is advantageous because it allows the doctor to slowly and controllably advance the distal opening 214 of the device 200 into the epidural space 60 in a safe manner. Next, a catheter may be inserted into the lumen 218 of the device 200, and exits through the opening 214 into the epidural space 60. The catheter is then used to deliver fluid, such as saline, pain medication, or any other drugs, into the epidural space 60. Alternatively, the device 200 itself may be used to deliver fluid, such as saline, pain medication, or any other drugs, into the epidural space 60. In some embodiments, while the doctor is advancing the tissue-cutting portion 210 through the ligamentum flavum 66, the proximal end of the handle 208 may be coupled to a fluid source (e.g., a syringe), and the doctor may operate the syringe to apply fluid delivery pressure within the lumen 218, as similarly discussed. Before the distal opening 214 exits pass the ligamentum flavum 66, the doctor will sense high pressure while operating the syringe. However, as soon as the distal opening 214 exits pass the ligamentum flavum 66 into the epidural space 60, the doctor will sense that the pressure associated with using the syringe has dropped, thereby knowing that the distal opening 214 has reached the epidural space 60.

In further embodiments, the device 10 itself may include the tissue-cutting portion 210 with the screw threads 212 and the distal opening 214 (FIG. 5). During use, after the doctor has placed the distal end 104 of the device 10 next to the ligamentum flavum 66, the doctor then rotates the device 210 about its longitudinal axis 230 to screw the tissue-cutting portion 210 into the ligamentum flavum 66. The doctor continues to advance the tissue-cutting portion 210 by screwing action until the opening 214 at the tissue-cutting portion 210 has passed through the ligamentum flavum 66 into the epidural space 60. Using the tissue-cutting portion 210 with the screw threads 212 to penetrate the ligamentum flavum 66 is advantageous because it allows the doctor to slowly and controllably advance the distal opening 214 of the device 10 into the epidural space 60 in a safe manner. Next, a catheter may be inserted into the lumen 107 of the device 10, and exits through the opening 214 into the epidural space 60. The catheter is then used to deliver fluid, such as saline, pain medication, or any other drugs, into the epidural space 60. Alternatively, the device 10 itself may be used to deliver fluid, such as saline, pain medication, or any other drugs, into the epidural space 60. In some embodiments, while the doctor is advancing the tissue-cutting portion 210 through the ligamentum flavum 66, the proximal end of the handle 108 may be coupled to a fluid source (e.g., a syringe), and the doctor may operate the syringe to apply fluid delivery pressure within the lumen 107, as similarly discussed. Before the distal opening 214 exits pass the ligamentum flavum 66, the doctor will sense high pressure while operating the syringe. However, as soon as the distal opening 214 exits pass the ligamentum flavum 66 into the epidural space 60, the doctor will sense that the pressure associated with using the syringe has dropped, thereby knowing that the distal opening 214 of the device 10 has reached the epidural space 60.

In the above embodiments described with reference to FIGS. 2-3, the audio device 140 is described as providing sound signal for indicating whether the device 10 is placed at a desired entry position and/or entry angle, or not. However, in other embodiments, in addition to, or instead of, providing the sound signal as described, the audio device 140 may provide audio information that assists the user in identifying the desired entry point and/or entry angle. For example, the audio device 140 may provide the distance-to-reference bodily structure, thereby informing the user the position of the device 10 relative to the reference bodily structure. The reference bodily structure may be a bony structure, such as a spinal process 78, or a non-bony structure, such as a ligamentum flavum 66 or an interspinous ligament 84.

In further embodiments, the audio device 140 may also provide navigation instruction for instructing the user how to maneuver the device 10 so that the device 10 is placed at a desired entry position and/or a desired entry angle. For example, the audio device 140 may audibly prompt the user to move the device 10 up, down, left, or right. The audio device 140 may also audibly prompt the user to tilt the device 10. In some cases, the audio device 140 may also audibly inform the user an amount of movement to be performed, such as a distance for which to move the device 10, and/or an angle for which to tilt the device 10. In some embodiments, the device 10 may further include an orientation sensing device (such as one or more accelerometers, or any of other orientation sensing devices known in the art) for determining an orientation of the device 10 relative to a reference coordinate. By comparing the actual orientation of the device with the desired orientation, the processor 130 can then determine an amount of tilting adjustment to be made. Also, in some embodiments, the processor 130 may use the reflected acoustic signal to estimate the configuration of the landscape within the body. From the estimated landscape configuration, the processor 130 may then determine an estimate of the desired entry trajectory. The processor 130 then calculates the location and/or orientation of the device 10 relative to the desired entry location and/or the desired entry orientation, and reports it to the user through the indicator 140. In some cases, as the user continuously reposition the device 10, the processor 130 continuously gather the reflected acoustic signals resulted from the different positioning of the device 10. The processor 130 may then use all of the reflected acoustic signals to estimate the configuration of the landscape within the body.

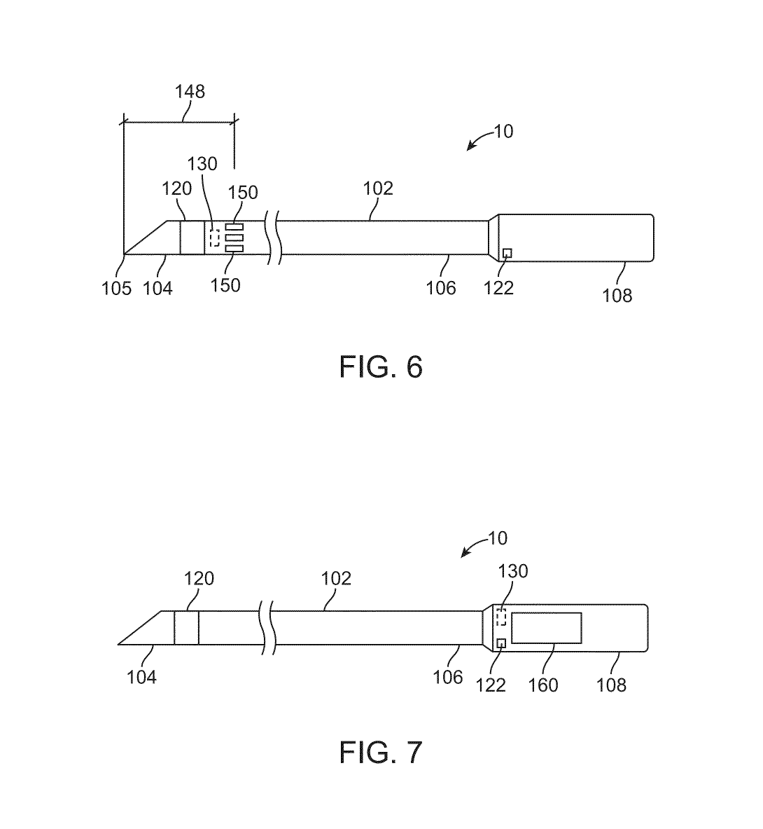

In the above embodiments, the indicator 140 has been described with reference to an audio device. However, in other embodiments, the indicator 140 may be implemented using other devices for providing other types of sensory signals. As shown in FIG. 6, instead of the audio device, the indicator 140 may be implemented using a plurality light sources 150. The light sources 150 may be implemented using LEDs, light bulbs, or any light emitting devices known in the art. In the illustrated embodiments, the light sources 150 are coupled to the processor 130 which provides activation signals for activating the light sources 150 during use. The light sources 150 are located closer to the distal end 104 than the proximal lend 160. This is advantageous because during use, the user's focus is near the distal end 104 of the device, and therefore, the user can see the light sources 150 without taking the focus away from the distal end 104 (which would otherwise be the case if the light sources 150 are located at the proximal end 106). In some embodiments, the light sources 150 may be located a distance 148 away from the distal tip 105 such that when the opening 109 at the distal end has reached the epidural space 60, the light sources 150 are still outside the patient. Such configuration allows the doctor to see the light sources 150 during the entire process of inserting the device 10 into the body. Also, as shown in the illustrated embodiments, the light sources 150 are disposed circumferentially around the shaft 102. This has the advantage that no matter how the device 10 is oriented, the user can see at least one of the light sources 150.

In some embodiments, the processor 130 is configured to activate the light sources 150, which assist the user in identifying a desired entry point, a desired entry angle, or both a desired entry point and a desired entry angle, for accessing the epidural space.

For example, in some embodiments, the light sources 150 are configured to provide the light signals when the distal end 104 of the shaft is at a desired entry location for accessing the epidural space 60. In other embodiments, the light sources 150 are configured to provide the light signals when the distal end 104 of the shaft is at a desired entry angle for accessing the epidural space 60. In further embodiments, the light sources 150 are configured to provide the light signals when the distal end 104 of the shaft is both at a desired entry location and at a desired entry angle for accessing the epidural space 60. The light signals provided by the light sources 150 may be green in color, and may remain on or may blink when the distal end 104 of the shaft 102 is at a desired entry location and/or desired entry angle. In other embodiments, the light sources 150 may provide light signals in other colors.

In other embodiments, the light sources 150 are configured to provide the light signals when the distal end 104 of the shaft is not at a desired entry location for accessing the epidural space 60. In other embodiments, the light sources 150 are configured to provide the light signals when the distal end 104 of the shaft is not at a desired entry angle for accessing the epidural space 60. In further embodiments, the light sources 150 are configured to provide the light signals when the distal end 104 of the shaft is both not at a desired entry location and not at a desired entry angle for accessing the epidural space 60. The light signals provided by the light sources 150 may be red in color, and may remain on or may blink when the distal end 104 of the shaft 102 is not at a desired entry location and/or not at a desired entry angle. In other embodiments, the light sources 150 may provide light signals in other colors.

In some embodiments, each light source 150 is configured to provide light signal in one color. In other embodiments, each light source 150 is configured to provide light signals in more than one color. In such cases, the processor 130 may selectively activate the light sources 150 to emit light signals in a first color (e.g., green color) when the distal end 104 of the shaft 102 is at a desired entry location and/or a desired entry angle, and may selectively activate the light sources 150 to emit light signals in a second color (e.g., red color) when the distal end 104 of the shaft 102 is not at a desired entry location and/or a desired entry angle.

In further embodiments, the light sources 150 may have a first subset of light sources 150 that emit light signals in a first color (e.g., green color), and a second subset of light sources 150 that emit light signals in a second color (e.g., red color). The first and second subsets of light sources 150 may be staggered relative to each other, and may be collectively disposed circumferentially around the shaft 102 in a ring configuration. In such cases, the processor 130 may selectively activate the light sources 150 in the first subset to emit light signals in a first color (e.g., green color) when the distal end 104 of the shaft 102 is at a desired entry location and/or a desired entry angle, and may selectively activate the light sources 150 in the second subset to emit light signals in a second color (e.g., red color) when the distal end 104 of the shaft 102 is not at a desired entry location and/or a desired entry angle.

In further embodiments, the light sources 150 may be used to inform the user the position of the device 10 relative to a reference bodily structure. For example, in some embodiments, when processor 130 determines that the distal end 104 of the shaft 102 relative to the reference bodily structure has reached a prescribed value, the processor 130 then activates the light sources 150 (e.g., by causing them to stay on or to blink). The reference bodily structure may be a bony structure, such as a spinal process 78, or a non-bony structure, such as a ligamentum flavum 66 or an interspinous ligament 84.

In some embodiments, the light sources 150 for informing the user of the position of the device 10 may be the same light sources 150 for indicating to the user that the desired entry location and/or entry angle has been reached or not. For example, the light sources 150 may blink when the desired entry location and/or entry angle has been reached (or not), and the same light sources 150 may stay on when the position device 10 relative to a reference bodily structure has been reached in some embodiments. In other embodiments, the light sources 150 may stay on when the desired entry location and/or entry angle has been reached (or not), and the same light sources 150 may blink when the position device 10 relative to a reference bodily structure has been reached in some embodiments. In further embodiments, another set of one or more light sources may be dedicated for informing user of the position of the device 10 relative to a reference bodily structure.

In further embodiments, the light sources 150 may be activated for instructing the user how to maneuver the device 10 so that the device 10 is placed at a desired entry position and/or a desired entry angle. For example, the processor 130 may selectively activate one of the light sources 150 (e.g., stays on) to instruct the user to move the device 10 towards a certain direction that corresponds with the angular position of the activated light source. The light sources 150 may also prompt the user to tilt the device 10. For example, the processor 130 may selectively activate one of the light sources 150 (e.g., blinks) to instruct the user to tilt the device 10 towards a certain direction that corresponds with the angular position of the activated light source. In other embodiments, the above activation configuration of the light source 150 may be reversed—i.e., the light source 150 may blink to instruct the user to move the device 10, and may stay on to instruct the user to tilt the device.

In some embodiments, the device 10 may have an orientation sensing device for determining an orientation of the device 10 relative to a reference coordinate, as similarly discussed. The orientation sensing device may also be configured to determine an angular orientation of the device 10 relative to its longitudinal axis. This allows angular positions of the light sources 150 to be established with respect to a reference coordinate, so that the processor 130 can accurately activate one of the light sources 150 for instructing the user how to position the device 10. For example, if the processor 130 determines that the device 10 needs to be moved up, the processor 130 then activates the corresponding light source 150 that is angularly located at the “top” of the device 10 (e.g., when viewed from a cross-sectional perspective).

In the above embodiments, the device of FIG. 6 has been described with reference to having a plurality of light sources. In other embodiments, instead of using a plurality of light sources 150, the indicator 140 may be implemented using a single light source 150. For example, in some embodiments, the light source 150 may have a ring configuration that is disposed around the shaft 102. Such configuration allows the user to see the light source 150 no matter how the device 10 is oriented. In some cases, the light source 150 may be implemented using a plurality of LEDs that collectively form a continuous ring configuration.

In other embodiments, the indicator 140 may be implemented using a display panel 160, such as a LCD display (FIG. 7). In the illustrated embodiments, the display panel 160 is located at the handle 108. In other embodiments, the display panel 160 may be located at the shaft 102, or may be communicatively coupled to the shaft 102 or the handle 108 via a cable or a wireless transmitter. The display panel 16 is coupled to the processor 130, which provides data for displaying information in the display panel 16. In some embodiments, the processor 130 may be configured to display an indicator in the display panel 16, which assists the user in identifying an entry point, an entry angle, or both an entry point and an entry angle, for accessing the epidural space.

For example, the display panel 16 may display an indicator when the distal end 104 is at a desired entry point, a desired entry angle, or both a desired entry point and a desired entry angle, for accessing the epidural space. The indicator may be “advance”, “OK”, “O”, or other indicators that inform the user that a desired entry point and/or angle has been achieved.

In other embodiments, the display panel 16 may display an indicator when the distal end 104 is not at a desired entry point, a desired entry angle, or both a desired entry point and a desired entry angle, for accessing the epidural space. The indicator may be “stop”, “X”, or other indicators that inform the user that a desired entry point and/or angle has not been achieved.

In further embodiments, the display panel 16 may display other information that assist the user in identifying the desired entry point and/or entry angle. For example, the display panel 16 may display distance-to-reference bodily structure, thereby informing the user the position of the device 10 relative to the reference bodily structure. The reference bodily structure may be a bony structure, such as a spinal process 78, or a non-bony structure, such as a ligamentum flavum 66 or an interspinous ligament 84.

In further embodiments, the display panel 16 may also display navigation instruction for instructing the user how to maneuver the device 10 so that the device 10 is placed at a desired entry position and/or a desired entry angle. For example, the display panel 16 may prompt the user to move the device 10 up, down, left, or right. The display panel 16 may also prompt the user to tilt the device 10. In some cases, the display panel 16 may also inform the user an amount of movement to be performed, such as a distance for which to move the device 10, and/or an angle for which to tilt the device 10. The device 10 may have an orientation sensing device for determining an orientation of the device 10 relative to a reference coordinate, as similarly discussed.

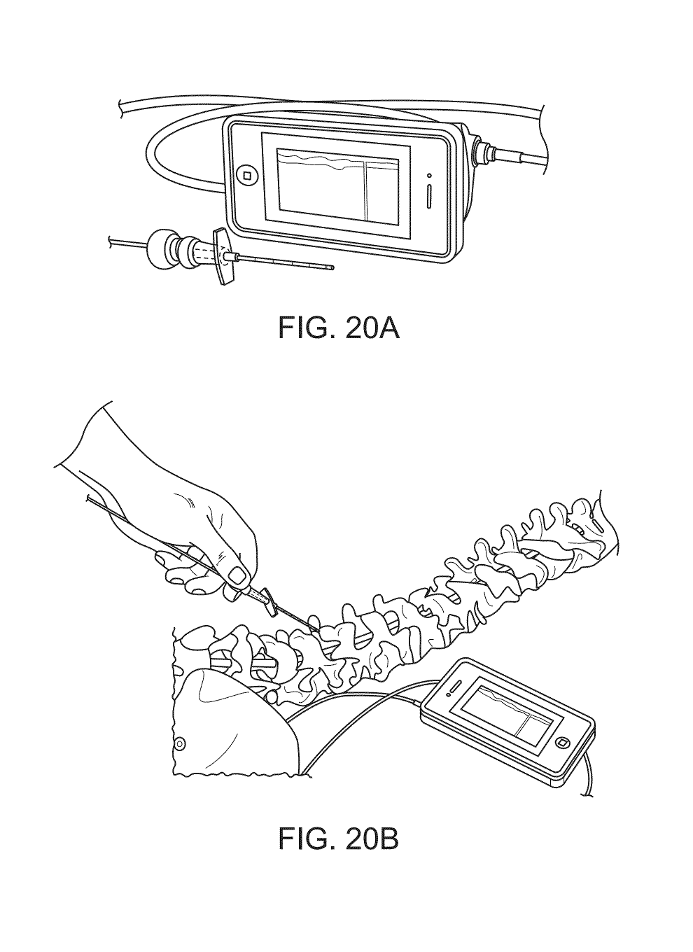

In other embodiments, the indicator 140 may be a separate device that is communicatively coupled to the device 10. For example, in other embodiments, the indicator 140 may be implemented using a peripheral device, such as a phone (e.g., iPhone—see FIGS. 20A-20B), or a PDA (e.g., a blackberry). In such cases, the device 10 includes a transmitter, such as a wireless transmitter, or a cable, for transmitting information to the peripheral device. The peripheral device may be configured to provide signal or information (such as any of the information described herein) for assisting the user in determining an entry location and/or entry angle of the device 10 for accessing the epidural space 60.

In further embodiments, the indicator 140 may include a tactile feedback mechanism at the handle 108 for informing the user of whether the device is at a desired entry location and/or entry angle, or not.

Also, it should be noted that any of the indicating techniques/features described with reference to some embodiments may be combined with other embodiments. For example, in other embodiments, the device 10 may provide both audio signal and visual signal (using light sources or a display panel) for assisting the user in identifying the desired entry point and/or the desired entry angle for accessing the epidural space 60.

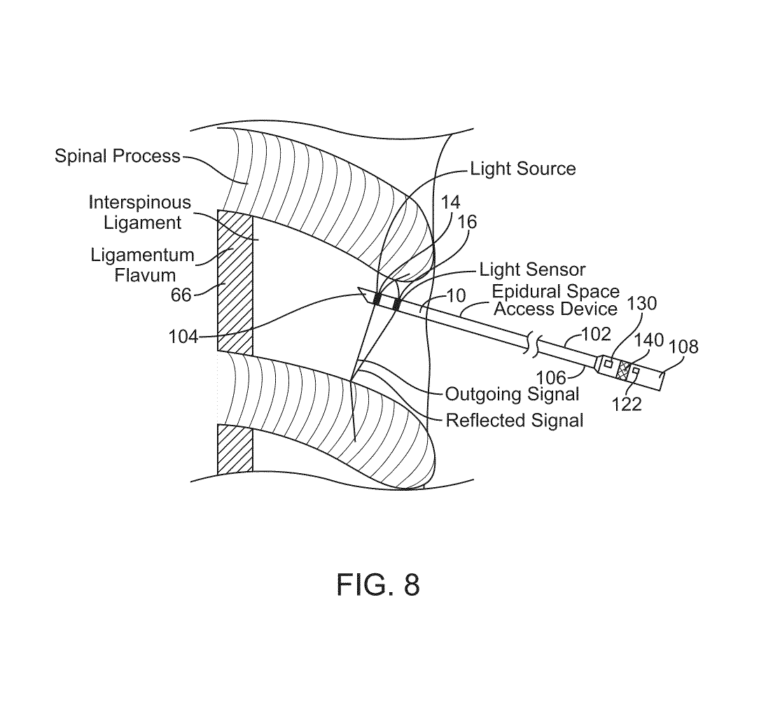

It should be noted that the technique for determining the desired entry trajectory should not be limited to using acoustic signals as described in the above embodiments, and that other techniques may be used in other embodiments. For example, in other embodiments, instead of the transducer 120, the device 10 may include a light emitting element 14 on the surface or internally contained within the device 10 (FIG. 8). Examples of the light emitting element include: light emitting diodes (LED), high power light emitting diodes (HPLED), organic light emitting diodes (OLED), quantum dot light emitting diodes, lasers, or other high intensity light transmitted by fiber optics, rod lens, or other light conductive means known to those of ordinary skill in the art. The light emitted from the element 14 is not limited to a specific frequency of visible or non-visible light. In other embodiments, instead of emitting light of certain wavelength or certain range of wavelengths, the light emitting device 14 may emit lights with different wavelengths (or colors in the case that the lights are visible) or ranges of wavelengths. This light emitting element 14 works independently or in combination with a sensing mechanism 15 (e.g., a light sensor) that measures reflected light. In some cases, the sensing mechanism 15 may include one or more fiber optics that carry light signals from the distal end to the proximal end of the device 10. The sensing mechanism 15 is communicatively coupled to the processor 130, which is configured to generate a signal to activate the indicator 140 in response to an input received from the sensing mechanism 15. In some embodiments, if a plurality of lights with different frequencies is used, the device 10 may be configured to simultaneously emit multiple lights in different frequencies, and simultaneously sense multiple reflected lights. Alternatively, the device 10 may be configured to sequentially emit multiple lights one at a time, and sense the reflected lights one at a time. In the illustrated embodiments, the processor 130 is configured to determine the opacity or density of surrounding tissue based on the reflected light signal received at the sensing mechanism 15. Various techniques may be used by the processor 130 for such purpose. For example, the processor 130 may be configured to determine the opacity or density of tissue by processing the light signal received by the sensing mechanism 15 to determine reflection coefficient and/or scatter coefficient of the tissue. From the opacity or density of surrounding tissue, the processor 130 may then determine whether the device 10 is at a desired entry point and/or a desired entry angle, or not. For example, if the reflected light signal indicates that the tissue (from which the light signal reflects) has a high density, then the processor 130 may determine that the distal end 104 of the device 10 is closer to the spinal processes 78 than the interspinous ligament 84 (like that shown in FIG. 3A), in which case, the processor 130 then determines that the device 10 is not at a desired entry position. Also, from the opacity or density of the surrounding tissue, the processor 130 may also calculate proximity of the distal end 104 of the device 10 relative to a reference bodily structure, such as a bony or dense structure, or a non-bony structure. In the illustrated embodiments, the sensing mechanism 15 is a component of the epidural space accessing device 10. In other embodiments, the sensing mechanism 15 is a separate, independent component. Also, in further embodiments, the device 10 may not include the sensing mechanism 15. In such cases, the reflected light may be sensed by user's eyes, by a separate device that includes a light sensor, or other devices that sense light using other techniques known to those skilled in the art.

In other embodiments, instead of or in addition to sensing reflected light, the sensing mechanism 15 may be configured to sense light scattered, absorbed, or emitted from a tissue.

In other embodiments, the device of FIG. 8 may include other types of indicators 140, such as one or more light sources 150, a display panel 160, or a tactile feedback mechanism, as similarly discussed with reference to the embodiments of FIGS. 6 and 7. Also, in other embodiments, the device of FIG. 8 may be used with, or may include, the device 200 as similarly discussed with reference to FIG. 4. Furthermore, in other embodiments, the device of FIG. 8 may have a tissue-cutting element 210 with threads 212 and a distal opening 214, as similarly discussed with reference to FIG. 5.

The manner of using the device of FIG. 8 is similar to that discussed with reference to FIGS. 3A-3D. In particular, during use, the device 10 is placed external to the patient and is used to emit light towards a patient's skin. Part of the light will be transmitted past the patient's skin towards an interior region inside the patient, and is then reflected from a bodily structure. The reflected light is then sensed by the sensing mechanism 15. Based on the reflected light sensed by the sensing mechanism 15, the processor 130 then determines an opacity or density of the bodily structure from which the light is reflected. If the opacity or density of the bodily structure indicates that the device is at a desired trajectory, the processor 130 then activates the indicator 140 to inform the user that the desired trajectory for accessing the epidural space 60 has been reached. If a desired trajectory has not been reached, the indicator 140 may emit a signal indicating such (or may remain silent), in which case, the user continues to reposition the device 10 until a desired trajectory has been reached. It should be noted that the processor 130 and the indicator 140 may be configured to inform the user that the desired entry trajectory has been achieved or not using other techniques in other embodiments, such as those described previously. Once the desired trajectory has been determined using the device 10, the user may then advance the device 10 along the trajectory to access the epidural space 60. After the distal end 104 of the device 10 has penetrated into the patient's skin, the device 10 may continue to emit light and sense reflected light, thereby allowing the user to know if the device 10 is being advanced correctly.

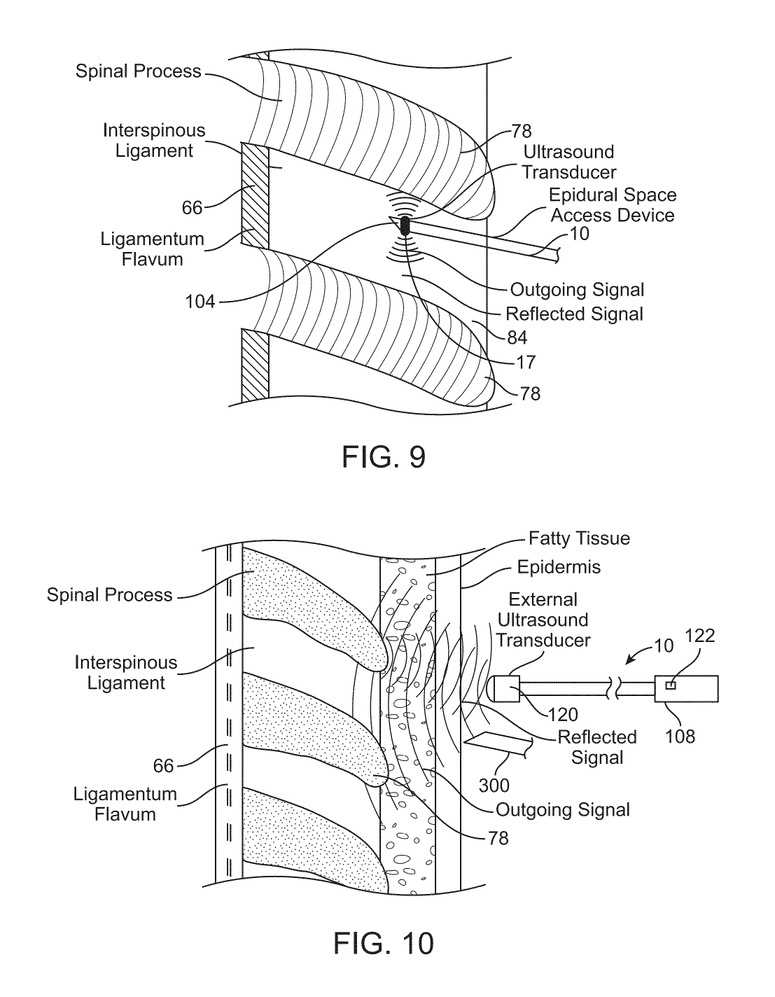

In further embodiments represented by FIG. 9, the epidural space accessing device 10 comprises a mechanism that takes advantage of differences in the mechanical impedance of soft tissue 84 and bone 78. For example, the epidural space accessing device 10 comprises an ultrasound transducer 17 (signal transmitter) at the distal end of the device 10 that uses ultrasound technology. Such technology functions by creating images for the examination of body organs by sending out high-frequency sound waves, which reflect off body structures. The processor 130 then receives these reflected waves and uses them to create a picture of internal bodily structures. Once the image of internal bodily structures is obtained, the processor 130 can then determine the position of the device 10 (or another device for accessing the epidural space 60) relative to the internal bodily structure(s). In other embodiments, the transducer 17 may be configured to emit sound waves in other frequency range.

In other embodiments, the device 10 may utilize other components known in the art to generate pressure waves. For example, the epidural space accessing device 10 may include a syringe needle with a plunger that is mechanically oscillated to generate pressure waves in liquid that comes into contact with tissue (e.g., tissue at and/or near the epidural space 60). The generated waves may have a low frequency (e.g., less than 60 Hz, and more preferably, less than 30 Hz), and may have a high amplitude. In some cases, the generated waves are considered as having a high amplitude when the waves are generated by displacing 0.1 to 1 cc of fluid, or more. During use, the syringe needle is inserted into the body, and the syringe plunger is mechanically oscillated (e.g., using a motor, such as a piezoelectric motor) to alternately push fluid (e.g., saline) out of the syringe needle, thereby creating low frequency waves. In such technique, the created low frequency high amplitude pressure waves are used to identify compliance (resistance to flow) of the surrounding tissue with respect to bone and other dense surrounding tissue. For example, if the tip of the needle is closer in proximity to bone or other dense tissue, the apparatus would have a greater impedance to inducing the oscillating fluid pressure than when the tip is located further away from bone or other dense tissue. By monitoring the oscillating backpressure in this manner, the user could identify the proximity of the tip to bony, dense or non-compliant biological structures. In some embodiments, the processor 130 is configured to cause an indicator 140 to generate a signal to inform the user of whether the device 10 is close to bony structure(s) or not. The indicator 140 may be any of the configurations described herein. In other embodiments, instead of using a syringe needle with a plunger, the device 10 may include other types of components for generating pressure waves.

In other embodiments, the device 10 may be configured to use the natural spatial periodicity of the spinal processes 78 to determine the location of the device 10 relative to reference bodily structure, such as a spinal process 78. In such cases, the device 10 may include a mechanism to create a resonant tone, and a disruptor for disrupting the resonant tone when the disruptor is physically placed between the spinal processes 78. In some cases, the disruptor may simply be a probe that is inserted between the spinal processes 78. The processor 130 is configured to detect a tonal change the disruptor causes, and uses the detected tonal change to determine the position of the device 10 between two processes 78. In other embodiments, the above technique may be employed to determine a position of an epidural space accessing device 10 between other bodily structures.

In other embodiments, the epidural space accessing device 10 may be configured to create an electric field (e.g., using electrodes) around the patient, around two specific spinal processes 78, or between bone or other dense structures. The electric field is simultaneously measured with one or more sensors at the epidural space accessing device 10 (or with another device having one or more sensors). In the illustrated embodiments, the epidural space accessing devices 10 use the principles of electrical impedance tomography (EIT), also known as applied potential tomography, to determine the position of the sensor element(s) relative to the surrounding bony or dense structures. EIT is a medical imaging technique in which an image of the conductivity or permittivity of part of the body is inferred from electrical measurements at tissue. During use, the conducting electrodes are placed against the skin (or other tissue) of the subject and small alternating currents are applied to some or all of the electrodes. The resulting electrical potentials are measured, and the process may be repeated for numerous different configurations of applied current. Once the image of the conductivity or permittivity of tissue around the epidural space 60 is obtained, the processor 130 can then determine the position of the device 10 (or another device) relative to the imaged tissue. Compared with techniques like computerized x-ray tomography and positron emission tomography, EIT is less costly approach, and requires no ionizing radiation. Further, EIT can produce thousands of images per second.

In other embodiments, the epidural space accessing device 10 may include a mechanism for identifying a location of a specific fluid. For example, in other embodiments, the device 10 may include signal transmitting device(s), such as piezoelectric crystals, ceramics or other transducers known to those of ordinary skill in the art, that transmit low frequency (e.g., less than 60 Hz, and more preferably, less than 30 Hz) sound waves toward the epidural space. In such cases, the device 10 (or another device) includes a sensor configured to measure the harmonics reflected back. The sensor is communicatively coupled to the processor 130, which analyzes the harmonics to identify the location of a specific fluid. In some embodiments, the detected fluid may be fluid in veins, arteries, spinal column, glands and other anatomical structures. By detecting the position of any or a combination of these fluids, the processor 130 may operate the indicator 140 to assist the user in avoiding these structures when inserting the epidural space accessing device 10. As a result, unnecessary harm may be prevented such as the inadvertent injection of analgesics or opioids as well as the inadvertent introduction of antigens that could lead to tissue infections, blood born infections or infections in the cerebral spinal fluid that are difficult to treat and could ultimately lead to death. In other embodiments, the location of the detected fluid containing anatomical structures may be used by the processor 130 to map regions of the anatomy that do not contain bony structures 78. As an example, the sensing mechanism and the processor 130 may be specifically configured to detect epidural veins and/or their contents (e.g., by identifying fluid flow region having certain size, shape, density, etc.) since epidural veins are present in the soft tissue leading up or surrounding the ligamentum flavum 66, but not in the surrounding bony structures 78. In further embodiments, the sensing mechanism is configured to sense cerebral spinal fluid (CSF), which is present just distal to the epidural space, for the purpose of setting the trajectory of the epidural space accessing device 10. By sensing these fluid containing anatomical structures, unnecessary harm may be prevented such as the inadvertent injection of analgesics or opioids as well as the inadvertent introduction of antigens that could lead to tissue infections, blood born infections or infections in the cerebral spinal fluid that are difficult to treat and could ultimately lead to death.

Other techniques may be used by the device 10 to detect presence of fluid. For example, in further embodiments, the epidural space accessing device 10 may be configured to detect the presence of fluid flow using the Doppler effect principle and ultrasound technology. The Doppler effect principle makes use of the apparent change in the frequency of waves occurring when the source and observer are in motion relative to each other, with the frequency increasing when the source and observer approach each other and decreasing when they move apart. This concept may be applied in a process that involves accessing the epidural space, where the presence of fluid flow through the epidural veins can be used to localize the ligamentum flavum 66 due to their presence in soft tissues but not in bony structures. In some cases, the processor 130 may use the position of the ligamentum flavum 66 to determine the position of the device 10 relative to the ligamentum flavum 66. If the processor 130 determines that the distal end 104 of the device 10 has moved past the ligamentum flavum 66, the processor 130 may then cause the indicator 140 to transmit a signal for indicating that the distal end 104 of the device 10 has reached the epidural space 60. Such technique has the benefit of obviating the need to use a syringe to apply a pressure for detecting a through-penetration of the ligamentum flavum 66 by the device 10.

Using similar techniques, the device 10 may be configured to detect cerebral spinal fluid (CSF) flow in some embodiments. Once the cerebral spinal fluid flow is detected, the processor 130 then uses the position of the cerebral spinal fluid to determine a desired trajectory (e.g., by referencing the trajectory off from the position of the cerebral spinal fluid) of the epidural space accessing device 10.

It should be noted that the techniques for identifying fluid near the epidural space are not limited to the examples described, and that any techniques, such as technique that involves use of ultrasound energy, or other types of energy, for detecting blood vessel location may be used by the device 10 to detect fluid near the epidural space. For example, any of the methods and/or devices disclosed in U.S. Pat. No. 5,259,385 may be used with embodiments described herein. U.S. Pat. No. 5,259,385 is hereby incorporated by reference as if repeated in its entirety herein.

In any of the embodiments described herein, the device 10 may not be the device that is directly used to access the epidural space 60. In such cases, the device 10 is not configured to puncture the patient's skin 80, and does not include the tip 105 for cutting (e.g., puncturing) tissue. During use, the device 10 is used to determine a desired entry position and/or entry angle for accessing the epidural space 60, and other device (such as a needle or an introducer sheath) is then used to puncture the patient's skin 80 to access the epidural space 60 using the entry position and/or entry angle determined using the device 10.

In other embodiments, the device 10 may be configured to assist determining of the desired entry point and/or entry angle using elastography. FIG. 10 illustrates an epidural space accessing device 10 that enables detection of a line-of-sight path through soft tissue to the ligamentum flavum 66 using elastography. The device 10 is used external to the body for assisting the user to identify a desired entry point and/or entry angle for accessing the epidural space 60 using another device 300 that is configured to directly access the epidural space 60 through the patient's skin. The device 10 can be used to avoid the intersection of the epidural space accessing device 300 with bone 78 prior to needle puncture. In the illustrated embodiments, the epidural space accessing device 10 includes a transducer 120 (such as a traditional “A” mode transducer) for determining an elastogram, which can then be used to identify soft tissue, and the relative location between the soft tissue and bony structures 78 up to a certain depth (depending on the design of the device 10). In certain embodiments, the relative location of the soft tissue and bony or dense structures are used to direct the user toward the correct position and angle with which to insert the epidural space accessing device 300. By knowing the relative location between the soft tissue and the bony or dense structures, the user can then place the epidural space accessing device 300 at the desired entry position and entry angle for insertion of the epidural space accessing device. In some cases, the correct angular orientation and point of epidural space accessing device entry is set by the device 10 at a location near the estimated point of epidural space accessing device insertion and at an orientation in-line with the approximate travel path of the epidural space accessing device 300. In other embodiments, instead of using the transducer 120 for use to determine the elastogram, other devices known in the art may be used to provide the elastogram. Also, it should be noted that the device 10 of FIG. 10 is not limited to having the transducer 120, and that in other embodiments, the device 10 may have other types of signal transmitter and/or signal sensor, such as those similarly discussed herein. Also, in further embodiments, the device 10 may include an indicator, such as any of the embodiments of the indicator 140 described herein, for providing signal(s) to assist the user in identifying the desired entry point and/or entry angle of the device 300. Furthermore, in other embodiments, the device 10 itself may be used to puncture the patient's skin 80 for accessing the epidural space 60. For example, in other embodiments, the device 10 may have a tissue-cutting tip (like the tip 105 shown in the embodiments of FIG. 2) for cutting tissue, and a lumen (like the lumen 107 shown in the embodiments of FIG. 2) for delivering another device (e.g., a catheter, a tissue-cutting device, etc.) or fluid (e.g., saline, pain medication, or other drugs).

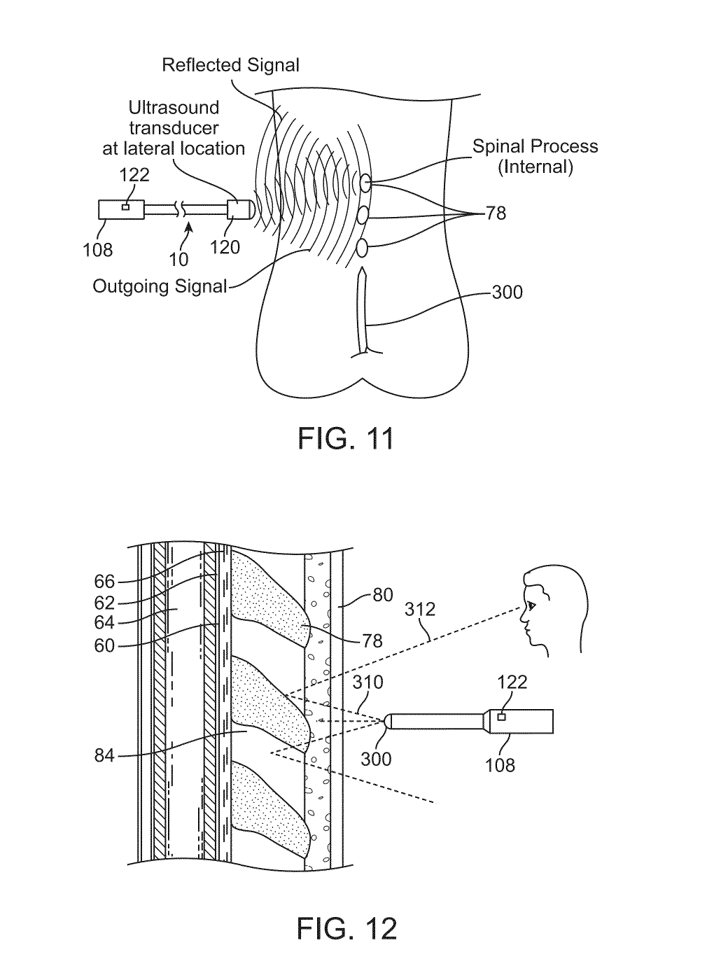

In other embodiments, any of the embodiments of the device 10 may be placed at other positions and is used to direct and/or sense signal from different directions. For example, in other embodiments, the device 10 with a transducer 120 (or other types of signal transmitter) can be used at a lateral location as generally shown in FIG. 11. This lateral location is generally perpendicular to the entry path of the epidural space accessing device 300. Such technique takes advantage of the flatter surface features of the spinal processes 78 encountered from this angle to provide an image to assist in determining the desired entry position and/or entry angle of the epidural space accessing device 300. In some embodiments, the image may be provided to a user for allowing a user to determine a desired entry point and/or entry angle of the epidural space accessing device 300 to access the epidural space 60. For example, using the image, the user may determine the relative location of soft tissue and bony structures up to a certain depth (depending on the design of the device 10). By knowing the relative location between the soft tissue and the bony or dense structures, the user can then place the epidural space accessing device 300 at the desired entry position and entry angle for insertion of the epidural space accessing device 300. It should be noted that the device 10 of FIG. 11 is not limited to having the transducer 120, and that in other embodiments, the device 10 may have other types of signal transmitter and/or signal sensor, such as those similarly discussed herein. Also, in further embodiments, the device 10 may include an indicator, such as any of the embodiments of the indicator described herein, for providing signal(s) to assist the user in identifying the desired entry point and/or entry angle of the device 300. Furthermore, in other embodiments, the device 10 itself may be used to puncture the patient's skin 80 for accessing the epidural space 60. For example, in other embodiments, the device 10 may have a tissue-cutting tip (like the tip 105 shown in the embodiments of FIG. 2) for cutting tissue, and a lumen (like the lumen 107 shown in the embodiments of FIG. 2) for delivering another device (e.g., a catheter, a tissue-cutting device, etc.) or fluid (e.g., saline, pain medication, or other drugs).

In any of the embodiments described herein, the device 10 may not include any sensor. Instead, the device 10 may include one or more transmitters, and no sensor. FIG. 12 illustrates another device 10 having a transmitter 300 in accordance with some embodiments. The device 10 also includes a handle 108 for allowing a user to position the device 10, and a control 122 for operating the transmitter 300 during use. The transmitter 300 is a light source configured to transmit light that has a certain prescribed wavelength or range of wavelengths, which may or may not be in the visible spectrum. During use, the user positions the device 10 by manipulating the handle 108 so that it is next to a patient's skin 80. The device 10 may be spaced away from the patient's skin 80 as shown, or alternatively, be abutted against the skin 80. The user than operates the control 302 to cause the transmitter 300 to emit light 310 towards the skin 80. At least some of the lights is transmitted pass the patient's skin 80 into an internal region of the patient, and is reflected by internal bodily structure. The reflected light 312 is transmitted out of the patient, and may be sensed by a user's eyes, or another device with a light sensor, in some embodiments. In some embodiments, the transmitter 300 is configured to transmit light having a certain wavelength (or range of wavelengths) such that a light reflected from within the patient may be directly sensed by a user to allow the user to determine a desired entry point and/or entry angle for accessing an epidural space. In such cases, the reflected light may have a feature (e.g., color, intensity, size, shape etc.), which is indicative of the configuration of the internal bodily structures, thereby allowing the user to see at least part of the internal bodily structures in real time on the patient's skin. In some embodiments, the user may reposition the device 10 using the handle 108 to direct light from the transmitter 300 towards other locations at the patient, thereby allowing the user to search for the desired entry location and/or entry angle.

In other embodiments, the device 10 may further include one or more filters for filtering light signals before the transmitted light reaches the patient. In further embodiments, the device 10 may include one or more filters for filtering light signals reflected from the patient. In still further embodiments, the device 10 may include respective filters for filtering light being transmitted towards the patient, as well as reflected light from the patient. In any of the embodiments described herein, the filter(s) for filtering light being transmitted from the transmitter 300 may be coupled to distal end of the device 10, or alternatively, be coupled to the skin of the patient (e.g., via an adhesive). Also, in any of the embodiments described herein, the filter(s) for filtering light being reflected from the patient may be coupled to the skin of the patient (e.g., via an adhesive), or alternatively, be coupled to another tool (such as a goggle, glasses, headwear, etc.) for use by the user.

In other embodiments, instead of having only one light source, the device 10 of FIG. 12 may include more than one light sources configured to emit respective lights having certain respective frequencies or range of frequencies. For example, one light source may be configured (e.g., to have a certain wavelength or range of wavelengths) to allow the user to detect spinal process 78, and another light source may be configured (e.g., to have a certain wavelength or range of wavelengths) to allow the user to detect interspinous ligament 84. Also, in further embodiments, instead of emitting lights, the transmitter 300 may be configured to emit other types of signals, such as any of the types of signals described herein, including but not limited to acoustic signals, ultrasound signals, magnetic signals, electric fields, etc. In such cases, the signals reflected back from the patient may be sensed using a sensor on a separate device. The sensed signals may then be processed by a processor, which is configured to assist the user in identifying a desired entry point and a desired entry angle for accessing an epidural space.

Also, in further embodiments, the device 10 of FIG. 12 may have a tissue-cutting tip, like the tip 105 shown in FIG. 2, which may be used to puncture the patient's skin in a process for accessing the epidural space. In such cases, after the distal end of the device 10 inserted into the patient, the transmitter 300 may continue to transmit light(s), which allows the user to continuously identify internal tissue structure (e.g., via reflected light signals transmitted from within the patient to outside the patient through the patient's skin) as the distal end of the device 10 is being advanced within the body.

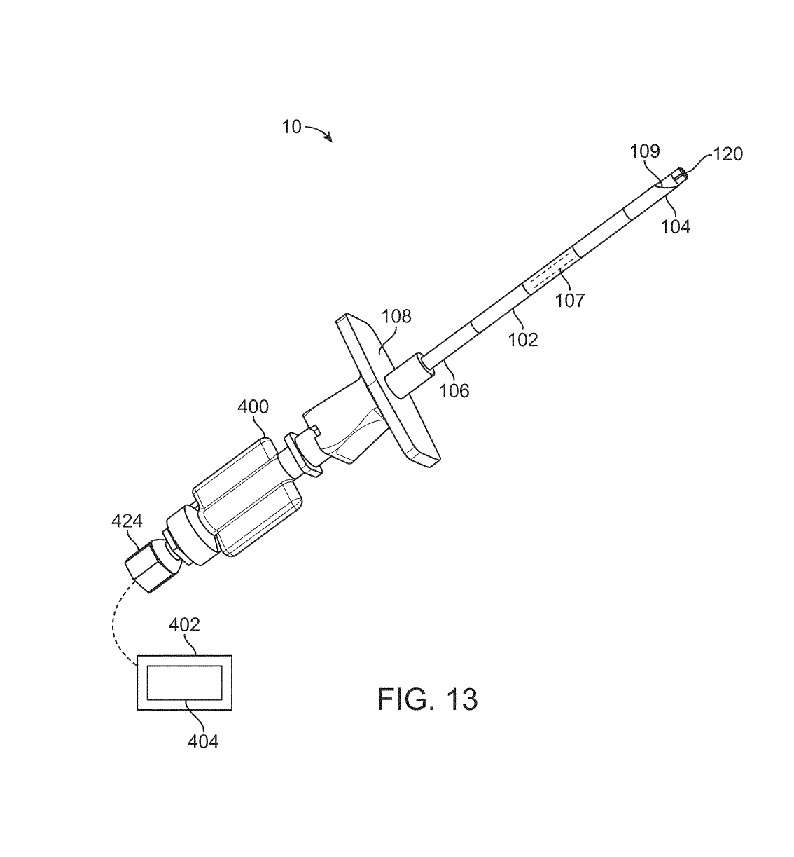

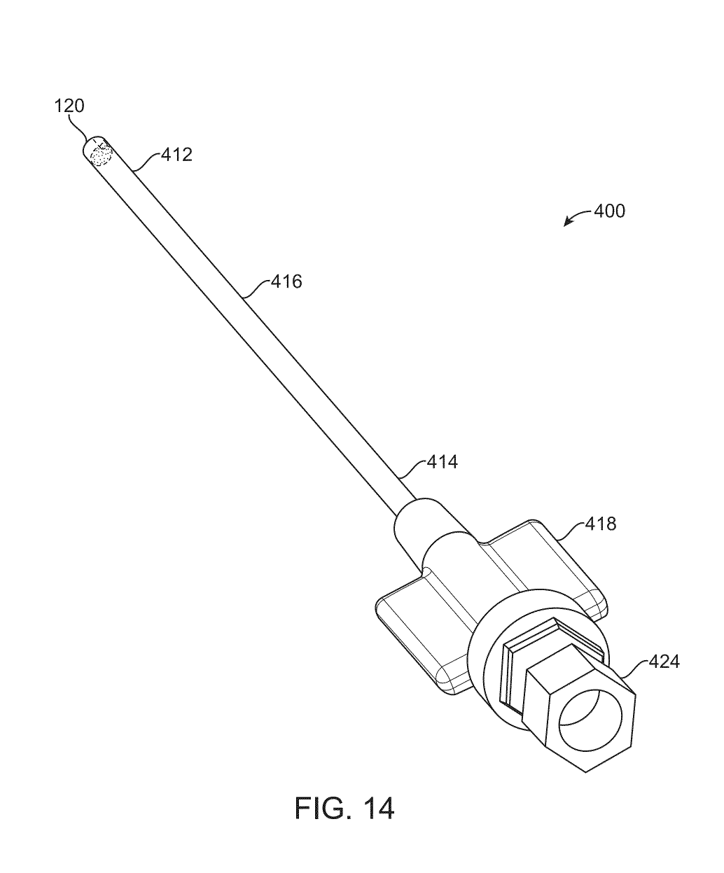

In the above embodiments of FIG. 4, the epidural space accessing device 10 is described as having a transducer 130 coupled to the shaft 102. However, in other embodiments, the transducer 130 may be coupled to another elongated member. FIGS. 13-15 illustrate an epidural space accessing device 10 in accordance with other embodiments, particularly showing a transducer component 130 coupled to a separate device that is different from the shaft 102. In particular, the device 10 includes a rigid shaft 102 having a distal end 104 and a proximal end 106, and a handle 108 coupled to the proximal end 106 of the shaft 102. The distal end 104 has a tapered tip 105. In some embodiments, the tapered tip 105 may be sharp for cutting (e.g., puncturing) tissue. The shaft 102 also has a lumen 107 extending between the distal end 104 and the proximal end 106, and a distal opening 109 that is in fluid communication with the lumen 107. The lumen 107 is for housing another device (such as a tissue cutting device, a catheter, etc.) and/or for delivering fluid into a body through the distal opening 109. In the illustrated embodiments, the epidural space accessing device 10 also includes a device 400 (which may be considered a component of the device 10) that includes a transducer component 120. In some embodiments, the transducer component 120 may be a one dimensional transducer array configured to emit ultrasound signals. In other embodiments, the transducer component 120 may be a two-dimensional transducer array configured to emit ultrasound signals.

The device 400 is configured (e.g., sized and/or shaped) for insertion into the lumen 107 of the shaft 102. In the illustrated embodiments, the device 400 may be detachably coupled to the shaft 102 by sliding distally into the lumen 107, and proximally out of the lumen 107. In some embodiments, after the device 400 is inserted into the lumen 107 of the shaft 102, the device 400 may be secured in position relative to the shaft 102 using a mechanical coupler. This ensures that the transducer component 120 stays at an operative position at the distal end 104 of the shaft 102. The mechanical coupler also allows the device 400 to be decoupled and removed from the shaft 102 after the device 400 has been used. In some embodiments, the mechanical coupler may be a latch mechanism that includes a latch and a portion to which the latch is detachably engaged. The latch may be located anywhere at the shaft 102 or handle 108, and the portion may be located at a corresponding position at the device 400, or vice versa. In other embodiments, the mechanical coupler may be a luer fitting. In one or more embodiments, the mechanical coupler may also function as a stopper for preventing the transducer component 120 from being advanced too far relative to the shaft 102.

As shown in FIG. 14, the device 400 includes an elongated member 416 having a distal end 412 and a proximal end 414, a handle 418 coupled to the proximal end 414, the transducer component 120 at the distal end 412, and a connector 424. In some embodiments, the elongated member 416 may be rigid. In other embodiments, the elongated member 416 may be flexible. The connector 424 is communicatively coupled to the transducer component 120 (e.g., through wire(s)). The transducer component 120 is configured to emit acoustic signals and to sense reflected acoustic signals. In response to the sensed reflected acoustic signals, the transducer component 120 generates output in the form of electrical signals. The output is transmitted to the connector 424, which in turn passes the output to the user interface 402. The user interface 402 (e.g., a processing unit in the user interface 402) processes the output from the transducer component 120, and generates visual indicators for presentation on the display 404. The processing unit for processing the output from the transducer component 120 may be implemented using circuit, such as a processor, in some embodiments.

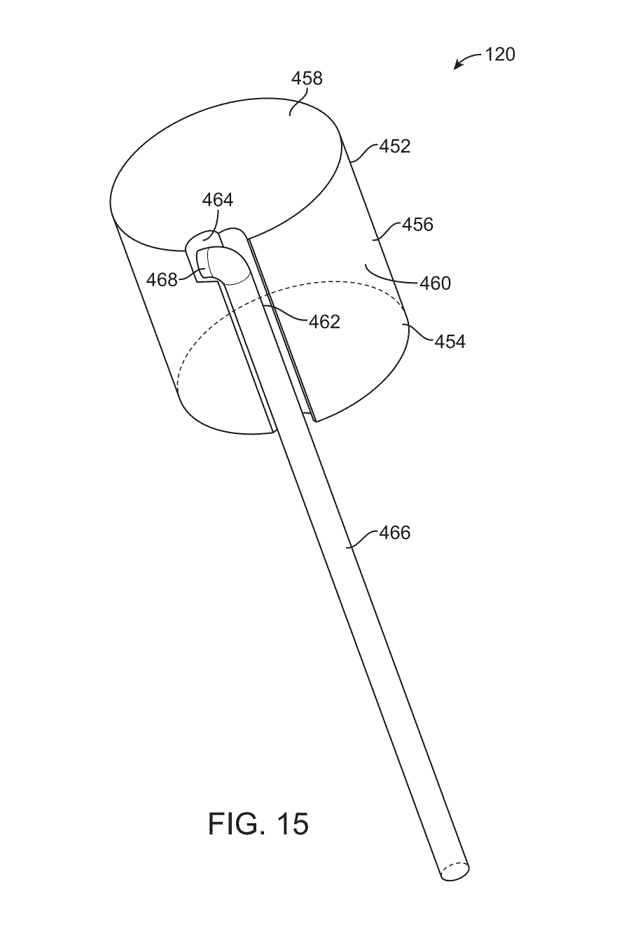

FIG. 15 illustrates the transducer component 120 in accordance with some embodiments. The transducer component 120 includes a distal end 452, a proximal end 454, and a body 456 extending between the distal end 452 and the proximal end 454. The transducer component 120 also includes an end surface 458 at the distal end 452, and a circumferential surface 460 defined by the body 456. The end surface 458 is forward looking so that the ultrasound signals may be emitted towards a distal direction from the end surface 458. The transducer component 120 further includes a first channel 462 at the circumferential surface 460, and a second channel 464 at the end surface 458. The first channel 462 is configured (e.g., sized and/or shaped) for accommodating a first segment 466 of a wire that connects the transducer component 120 to the connector 424, and the second channel 464 is configured (e.g., sized and/or shaped) for accommodating a second segment 468 of the wire that forms an angle relative to the first segment 466. In the illustrated embodiments, the angle between the first and second segments 466, 468 is 90°. In other embodiments, the angle may be more than 90° or less than 90°. Also, it should be noted that, as used in this specification, the term “channel” is not necessarily limited to a space having an elongated configuration, and may refer to any slot or opening having any dimension or shape. In the illustrated embodiments, after the segments 466, 468 of the wire are placed in the respective channels 462, 464, the distal end of the wire may then be secured to the transducer component 120. For example, a weld may be placed into the channel 464 to fill the space that is not occupied by the segment 468 and the bend of the wire, thereby providing a substantially coplanar surface with the end surface 458.

In some embodiments, an electrically-isolating coating may be applied to the body 456 to isolate the two electrodes on the distal and proximal portions of the piezoelectric material. The coating may also be applied to the end surface 458 which may further be used to protect the body from contact to materials used to fabricate the end surface 458 and/or the body 456. The coating may be made from materials such as epoxies, silicones, polymers, parylenes, or any of other similar coating materials. These materials may have an acoustic impedance lower than the materials beneath them if used on the end surface 458. In some embodiments, the coating may be formed from multiple layers of different materials. The thickness of the coating may be configured to form an acoustic matching layer, or layers, as to minimize internal acoustic reflections within the transducer component 120. In some embodiments, if the coating is an effective single matching layer, the thickness may be substantially a quarter-wavelength thickness (quarter-wavelength±10% of quarter-wavelength) relative to the fundamental transmit frequency used in the transducer component 120. In other embodiments, if the coating is part of a multiple layer acoustic matching design, the thickness may be less than a quarter wavelength thickness.

During use, the device 400 is communicatively coupled to a user interface 402, which provides visual indicators for presentation on the display 404 for allowing a user of the device 10 to determine a desired entry location and/or direction, and/or a desired trajectory based on signals provided by the transducer component 120. In the illustrated embodiments, the communicatively coupling between the device 400 and the user interface 402 may be achieved using the connector 424, to which a cable with one end may be attached, and with another end attached to the user interface 402. In other embodiments, the communicatively coupling between the device 400 and the user interface 402 may be achieved using a wireless component. For example, there may be a first wireless component for transmitting signals from the transducer component 120 to a second wireless component at the user interface 402, which receives the signals.

In some embodiments, the display 404 is configured (e.g., by a circuit inside the user interface 402) to provide visual indicators. In particular, the circuit in the user interface 402 is configured to receive output from the transducer component 120 (which provides the output based on reflected acoustic signals), and process the output to generate signals based on the output from the ultrasound component 120, the signals representing information (visual indicators) for presentation in the display 404. In some embodiments, the indicators indicate that the distal end 412 of the elongated member 416 (or the transducer component 120) is at a desired entry location for accessing the epidural space. In other embodiments, the display 404 is configured to provide the visual indicators to indicate that the distal end 412 of the elongated member 416 (or the transducer component 120) is at a desired entry angle for accessing the epidural space. In further embodiments, the display 404 is configured to provide the visual indicators to indicate that the distal end 412 of the elongated member 416 (or the transducer component 120) is both at a desired entry location and at a desired entry angle for accessing the epidural space.

In other embodiments, the display 404 is configured to provide the visual indicators to indicate that the distal end 412 of the elongated member 416 (or the transducer component 120) is not at a desired entry location for accessing the epidural space. In other embodiments, the display 404 is configured to provide the visual indicators to indicate that the distal end 412 of the elongated member 416 (or the transducer component 120) is not at a desired entry angle for accessing the epidural space. In further embodiments, the display 404 is configured to provide the visual indicators to indicate that the distal end 412 of the elongated member 416 (or the transducer component 120) is both not at a desired entry location and not at a desired entry angle for accessing the epidural space.

The user interface 402 may have different configurations in different embodiments. In some embodiments, the user interface 402 may be implemented using a handheld device, such as a phone, an iPhone (see, e.g., FIGS. 20A-20B), a tablet, an iPad, a Blackberry, or any of other handheld devices. In other embodiments, the user interface 402 may be a computer, such as a desktop or a laptop. In further embodiments, the user interface 402 may be implemented using a generator configured to provide control signals for driving the transducer component 120. In such cases, the generator may include the display 404 or providing visual signals to the user. In one or more embodiments, the user interface 402 may be considered to be a component of the device 400 and/or the device 10. In other embodiments, the user interface 402 may be considered to be a separate device from the device 400 and the device 10.

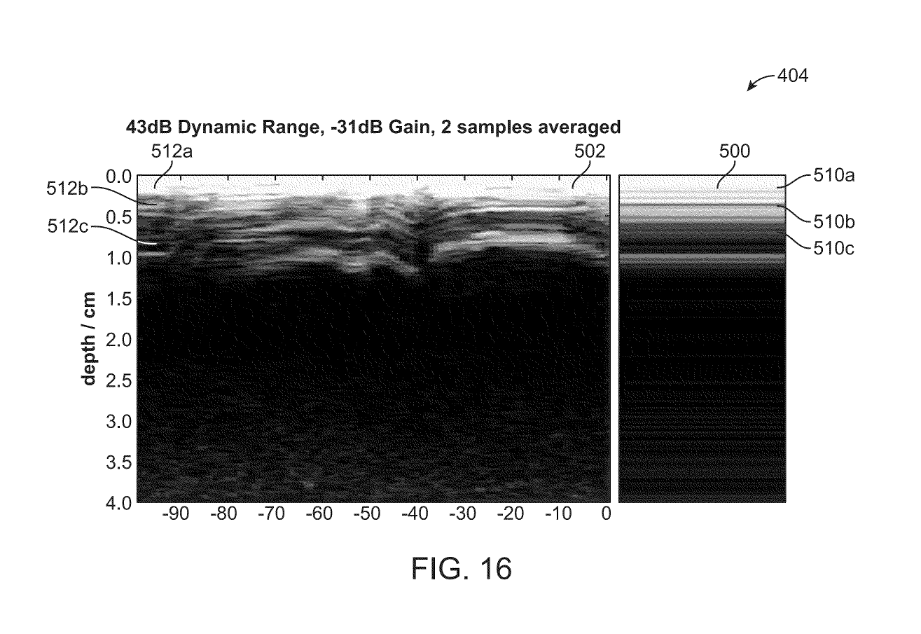

As shown in FIG. 16, in some embodiments, the visual indicators (signals) displayed in the display 404 may include first indicators 500 and second indicators 502. The first indicators 500 include a series of lines 510 (e.g., lines 510a-510c). Each of the lines 510 represents a corresponding intensity of the reflected acoustic signal in real time, and corresponds to a respective tissue depth (e.g., depth of tissue relative to a reference location, such as the location of the transducer component 120). The lines 510 are oriented horizontally in the illustrated embodiments, but in other embodiments, they may be oriented at other angles. The lines 510 are dynamically changing based on a positioning of the ultrasound component 120. In some embodiments, the user of the epidural space accessing device 10 may use the pattern of the lines 510 to determine whether the distal end of the epidural space accessing device 10 is being desirably positioned. For example, the user may realize that the pattern of the lines 510 indicates that the distal end of the epidural space accessing device 10 is pointing towards bone, and/or has not reached the ligamentum flavum. In another example, the user may realize that the pattern of the lines 510 indicates that the distal end of the epidural space accessing device 10 is pointing to a space between bones and/or has reached the ligamentum flavum.

In other embodiments, the user interface 402 may include a processing unit (e.g., circuit with or without software) configured to process the pattern of the lines 510, and determine whether the distal end of the epidural space accessing device 10 is desirably positioned. For example, the processing unit may be configured to identify certain line pattern (e.g., through template matching, and/or pattern analysis) that has been pre-determined as corresponding to a correct placement of the epidural space accessing device 10. The analysis of the line pattern may involve determining locations of the lines, widths of the lines, intensities of the lines, etc., or any combination of the foregoing. In some embodiments, when the processing unit has identified such line pattern based on the output from the transducer component 120, the processing unit may then inform the user that a correct placement of the epidural space accessing device 10 has been reached. In some embodiments, the processing unit may be configured to generate a visual signal (e.g., a green light) at the display 404 or a LED to indicate to the user that the epidural space accessing device 10 has been desirably positioned. In such cases, when the device 10 has not been desirably positioned, the LED or the display 404 may display a red light, or may not display any light signal.

In one implementation, the user interface 402 may include a non-transitory medium that stores a plurality of templates, with each template having a pattern that has been pre-determined as corresponding to a desired placement of the epidural space accessing device 10. In such cases, when the processing unit receives the real-time output from the transducer component 120, the processing unit processes the output to determine corresponding intensity lines (e.g., lines 510) in real-time, and compares the pattern formed by the lines 510 with the templates to see if a match can be found. If the processing unit finds a match, then the processing unit may determine that the device 10 has been desirably positioned, and may inform the user accordingly.

In further embodiments, the processing unit in the user interface 402 may be configured to identify bone versus soft tissue based on an intensity of the reflected acoustic signal. For example, in some embodiments, when the intensity of the reflected acoustic signal indicates that the reflected acoustic signal is from bone, then the processing unit may provide such indication in the display 404. In some embodiments, the processing unit may provide color coding to distinguish bone from soft tissue. For example, the processing unit may provide a green color for one or more of the lines 510 to indicate that they correspond to soft tissue, and/or may provide a red color for one or more of the lines 510 to indicate that they correspond to bone.

As shown in the figure, the second indicators 502 also include a series of line 512 (e.g., lines 512a-512c), each of the lines 512 corresponding to a respective tissue depth (e.g., depth of tissue relative to a reference location, such as the location of the transducer component 120). The lines 512 form a histogram that indicates a history of the navigation result. In particular, the lines 512 are presented in a graph in which the y-axis represents tissue depth, and the x-axis represents time. The lines 512 are moved across the graph (and therefore the display 404) in a right-to-left manner, so that the user can see a history of the line patterns based on previous maneuvers of the epidural space accessing device 10. Thus, as the epidural space accessing device 10 is being maneuvered, the histogram formed by the lines 512 is dynamically updated. In particular, at a certain point in time, the intensity lines 510 will form a certain pattern, and the pattern will be captured as a snap shot that shows up at the end of the lines 502. In other words, the pattern of the lines 512 at any point in time shown in the histogram corresponds to the pattern of the lines 510 at that point in time. Accordingly, the lines 512 (e.g., the respective ends of the lines 512) are dynamically changing based on a positioning of the ultrasound component 120. The lines 512 are oriented generally horizontally in the illustrated embodiments, but in other embodiments, they may be oriented at other angles.

In the illustrated embodiments, both indicators 500, 502 are displayed in the display 404. In other embodiments, the first indicators 500 are displayed in the display 404, and the second indicators 502 are not displayed. In further embodiments, the second indicators 502 are displayed in the display 404, and the first indicators 500 are not displayed.

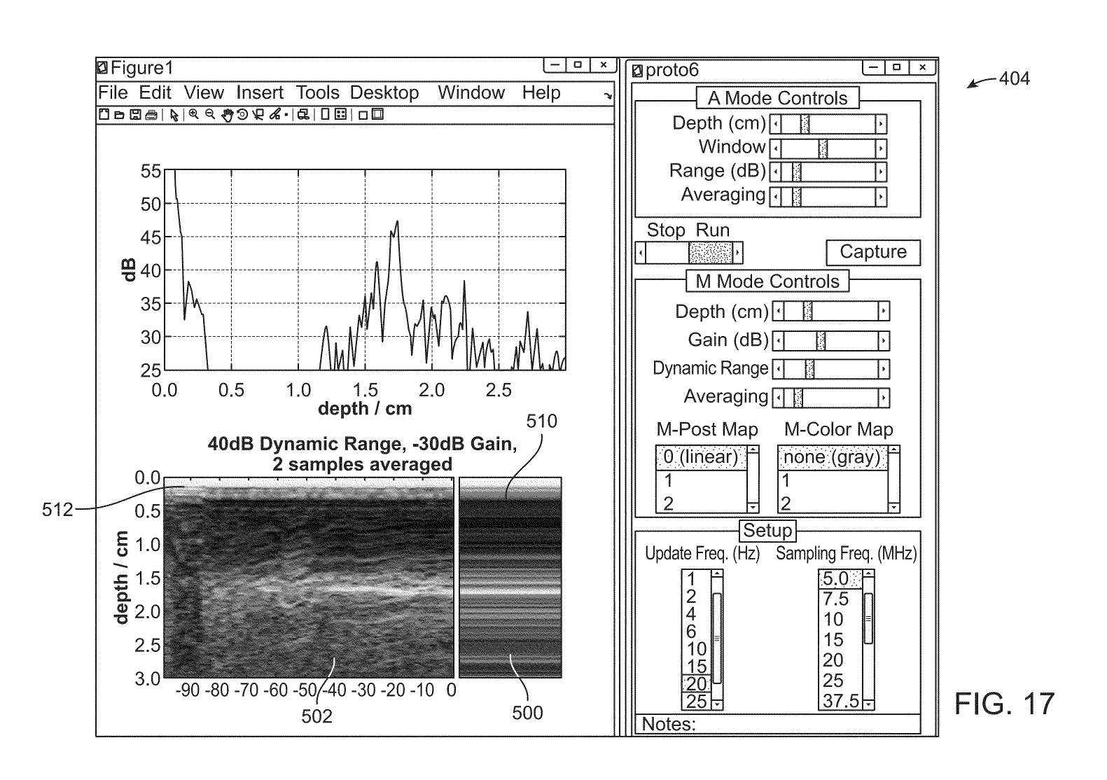

Also, in other embodiments, the display 404 may provide other information (FIG. 17). In the illustrated embodiments, the display 404 may be a graphical user interface showing outputs from an Amplitude mode (A-mode) and/or Motion mode (M-mode), thereby providing the user with information about the reflections or echoes of ultrasound from the tissues of interest. The display in the upper left part of the display 404 in FIG. 17 shows an example of A-mode. The horizontal axis is the distance into the interrogated tissue in centimeters (cm). The vertical axis is the amplitude of the returning echoes shown in decibels (dB). The high level signal around 1.7 cm is an example of a reflection from bone. The “A Mode Controls” in the upper right of the display 404 is a control for allowing a user to modify the inputs to the display as well as the display in the upper left of the display 404. The “Depth (cm)” control allows a user to control the maximum distance displayed into the tissue. The “Range (dB)” allows a user to control the maximum signal level shown, which is 55 dB in the illustrated example. The “Window” allows the user to control the lowest signal level display, which is 25 dB in the illustrated example. The “Averaging” control allows a user to adjust how many echoes are averaged together before they are displayed.

The display 404 in the lower left part of the user interface 402 shows an example of M-mode. The horizontal axis represents time in normalized units. The extent of the display may show a few seconds of signals or tens of seconds and may be adjustable by the user. The amplitudes of the A-mode signals may be color-coded with greyscale or color values and displayed as vertical lines on the M-mode display. Over time new vertical lines of amplitude information are displayed so the M-mode display scrolls from right to left as new vertical lines are added on the far right in the M-mode display. In some embodiments, the presentation of the information in the M-mode display occurs in real time. The vertical axis represents the distance into the tissue where the top of the display represents where a transducer is located. The “M Mode Controls” in the lower right of the display 404 allows a user to modify the inputs to the display as well as the display in the lower right of the display 404. The “Depth (cm)” allows a user to control the maximum distance into the tissue. The “Gain (dB)” allows a user to adjust the global signal level for all distances. The “Dynamic Range” allows a user to control the levels of amplitude signals shown in the display. For example, the display may show 40 decibels (decibel range) of information as seen in the illustrated figure. The single “Dynamic Range” control in the display 404 may be used by a user to control the minimum and maximum decibel levels defining the boundary of the decibel range. The “Averaging” control allows a user to adjust how many echoes are averaged together before they are displayed per vertical bin in the M-mode display. The user may select from several colorized maps through the “M-Color Map” control which may include greyscale as well as color maps. The relationship between signal amplitude and displayed pixel value (color or greyscale) may be controlled through the “M-Post Map” control. This control allows nonlinear relationships between increasing signal amplitude and increasing values in the selected color map be established.

The area in the lower right in the display 404 labeled “Setup” provides the user with control over the rate at which information is displayed on the user interface in units of Hertz through the “Update Freq. (Hz)” control. Higher frequencies update the display more frequently. The “Sampling Freq (MHz)” control provides the user with the ability to trade-off the amount of signal averaging that may be employed before signals are displayed with the spatial resolution of the signals in the A-mode and M-mode displays. Higher sampling frequencies provide improved spatial resolution, however the amount of averaging across A-mode lines of echoes is diminished. It should be noted that the values shown in the “Setup” section of the display 404 are examples, and that in other embodiments, the values may be different or have different ranges. For example, higher sampling frequencies may be employed in a different hardware implementation that would maintain acceptable spatial resolution without any compromise in acceptable amounts of signal averaging.

The “Stop” and “Run” controls in the far right column in the display 404 provide the user with the ability to run the system and stop the system when selected. The “Capture” button provides a mechanism for the user to instruct the system to save raw ultrasound digitized signals before any signal processing is applied. This mechanism allows the user to process signals on a computer after signal acquisition for further analysis.

In some embodiments, the user interface 402 may also be used to control the transducer component 120 (e.g., to turn the transducer component 120 “ON” or “OFF”, etc.). In such cases, the user interface 402 may include a button, a keyboard, a mic for receiving voice commands, a sliding mechanism, a touch screen, or any of other user input devices.

In one or more embodiments, the device 10 in the embodiments of FIG. 13 may optionally further include a speaker (e.g., the speaker 140) for providing audio signals to the user of the epidural space accessing device 10. The speaker may be located at the handle 108, at the handle 418 of the device 400, or at the user interface 402. In such cases, the speaker may provide the audio signals in accordance with the embodiments discussed with reference to FIG. 4.

Also, in one or more embodiments, the epidural space accessing device 10 in the embodiments of FIG. 13 may optionally further include the tissue cutting device 200 discussed with reference to the embodiments of FIG. 4. In some embodiments, the tissue-cutting device 200 may be configured (e.g., sized and/or shaped) for insertion into the lumen 107 of the rigid elongated member 102. In such cases, the lumen 107 is used to house the device 400 for assisting the user to navigate the distal end of the epidural space accessing device 10. When the distal end of the epidural space accessing device 10 is desirably positioned (e.g., next to the ligamentum flavum), the device 400 is then removed from the lumen 107, and the tissue-cutting device 200 is then inserted into the lumen 107. In other embodiments, instead of using the same lumen 107 for alternately accommodating the devices 200, 400, the elongated member 102 may include a first lumen 107 for accommodating the device 400, and a second lumen for accommodating the tissue-cutting device 200. In such cases, the device 400 for tissue navigation may be used simultaneously together with the tissue-cutting device 200.

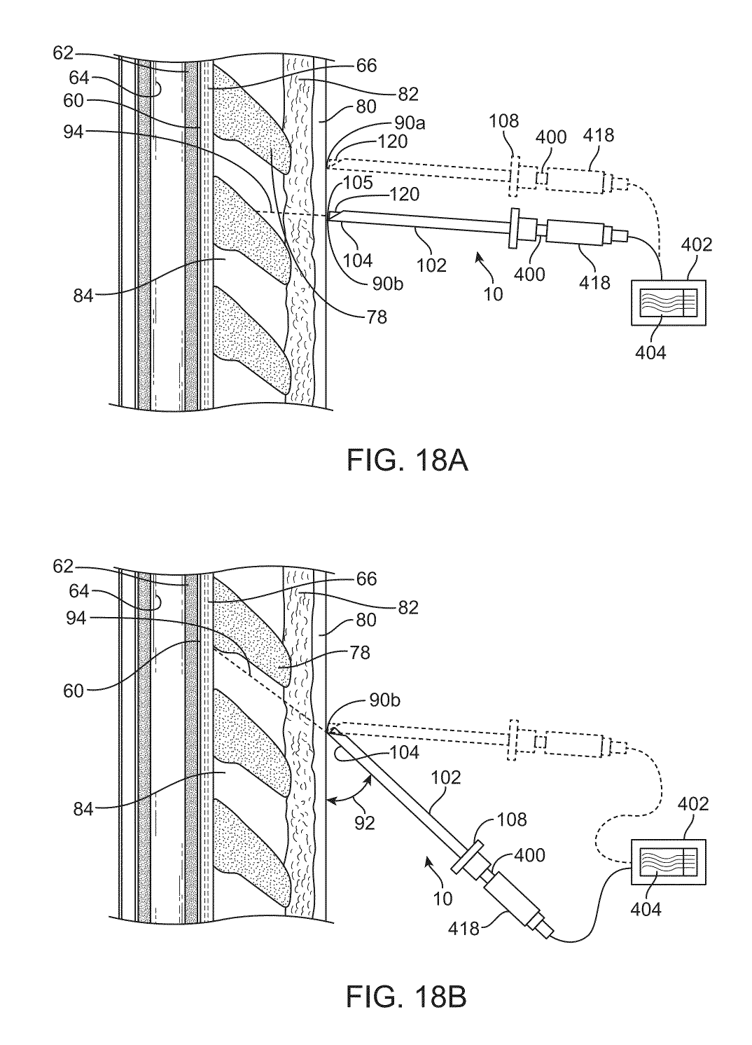

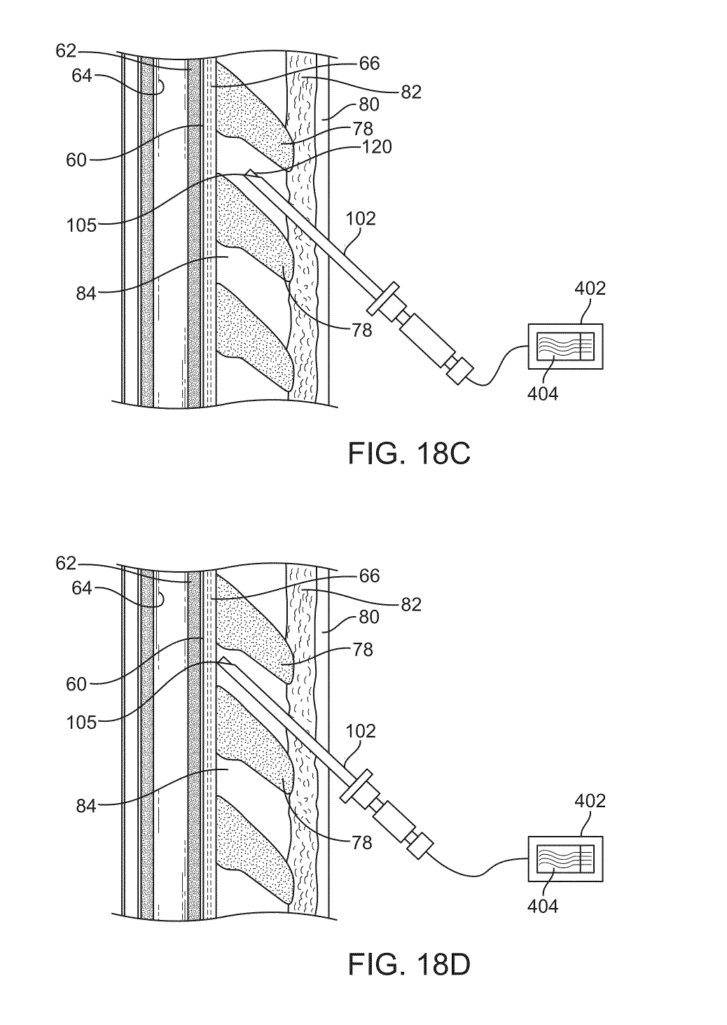

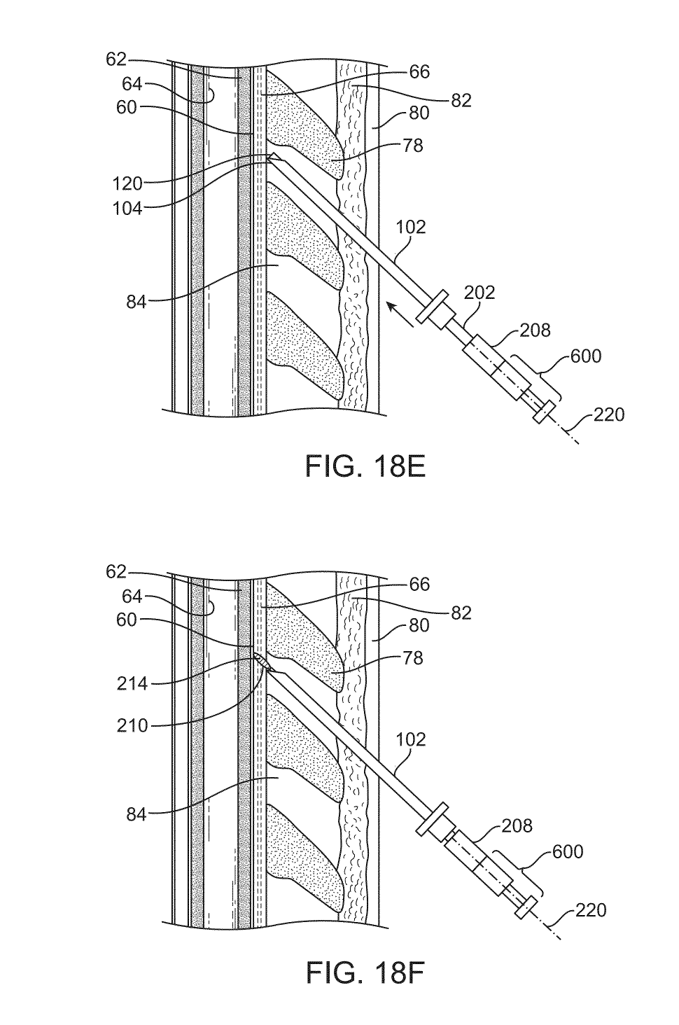

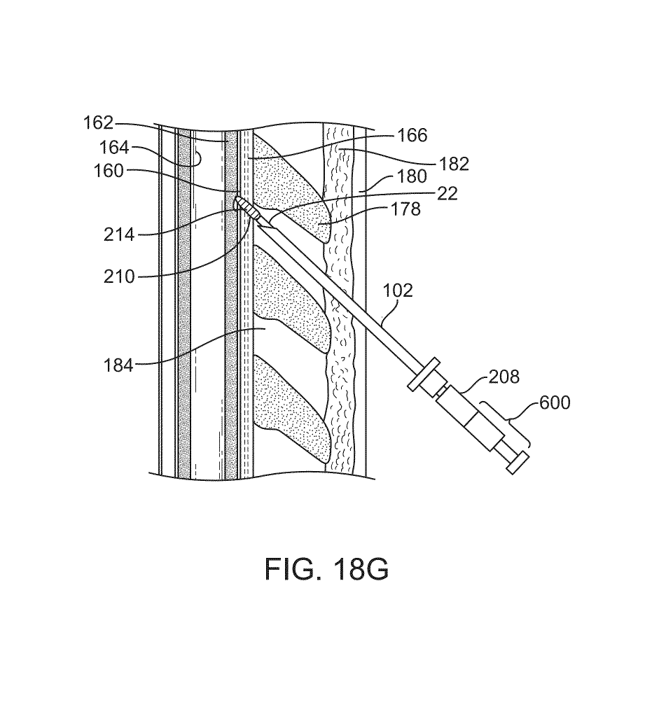

FIGS. 18A-18G illustrate a method of using the epidural space accessing device 10 of FIG. 13 in accordance with some embodiments. As shown in FIG. 18A, the tissues that define the epidural space 60 including the dura mater (or dura 62) which is a protective covering that sheaths the spinal cord 64, the ligamentum flavum 66 which is a ligament adjacent to the dura 62 that runs longitudinally along the spinal column, and the bony sides of the vertebral canal. To access the epidural space 60, the patient is positioned either seated or on his/her side, and is instructed to flex the back outward to maximize the spacing between the outer vertebral components. The spinal processes 78 are palpated, and the interlaminar space is estimated. In order to reach the epidural space 60, the distal end 104 of the shaft 102 will need to penetrate the skin 80, soft tissue 82, and interspinous ligament 84.

Before the epidural space accessing device 10 is used to penetrate the skin 80, the doctor (user) may use the device 400 to determine an entry point and an entry orientation (which collectively define an entry trajectory/path) for the epidural space accessing device 10 at the patient's skin 80. In particular, the doctor may insert the device 400 into the lumen 107 of the shaft 102, so that the transducer component 120 is at the distal end 104 of the shaft 102. The doctor may then place the epidural space accessing device 10 at a location that the doctor believes is close to a desired entry point. For example, the doctor may place the distal end 104 at location 90a (as represented by the dashed object in FIG. 18A). The transducer 120 is then activated using a control at the user interface 402 to emit an acoustic signal towards an interior region in the body. In some cases, to provide or improve the acoustic coupling between the transducer component 120 and the patient's skin 80, the transducer component 120 may be located at the tip of the epidural space accessing device 10, and/or the patient's skin 80 may be applied with a layer of gel. Alternatively, the distal tip 105 of the epidural space accessing device 10 may be inserted slightly into the patient's skin 80 (e.g., placed within the skin layer 80 or within the tissue layer 82) to allow the transducer component 120 to acoustically couple to the patient's skin. The transducer component 120 is also used to sense signal reflected back from within the interior region in the body. In the example shown, because the distal end 104 at position 90a is close to the spinal process 78, the acoustic signal transmitted from the transducer component 120 will be reflected back from the spinal process 78, and be sensed by the transducer component 120.

In the illustrated embodiments, based on the reflected acoustic signal, the processing unit generates visual indicators for presentation at the display 404. The doctor may use the visual indicators in the display to decide whether a desired position of the epidural space accessing device 10 has been achieved. In some embodiments, the processing unit in the user interface 402 is configured to determine the time it takes for a transmitted signal to be reflected back to the transducer component 120. In the example, because of the close proximity between the spinal process 78 and the transducer component 120, the time it takes for the signal to be transmitted and reflected back will be relatively short. Also, the reflected signal may have an amplitude that is relatively strong because the reflected signal is not significantly attenuated by much soft tissue before it reaches back to the transducer component 120 (or another sensing mechanism). As such, using the timing information and/or the amplitude information, the processing unit may determine that the current position 90a of the distal end 104 is close to bone 78, and is therefore not desirable for accessing the epidural space. Accordingly, the processing unit does not activate any indicator (e.g., LED indicator, speaker, etc.) to emit a signal to indicate that a desirable entry location has been reached. Alternatively, the processing unit may cause the indicator to emit a signal to let the doctor know that a desired entry position has not been reached.

Upon knowing that a desired entry trajectory has not been reached, the doctor continuously repositions the epidural space accessing device 10. When the distal end 104 is at position 90b, the transducer component 120 is further away from the bony structures. The entry position 90b for the distal end 104 is better than the entry position 90b, because at entry position 90b, the distal end 104 may be advanced further into the patient without hitting bone 78. However, the epidural space accessing device 10 may still not be in an ideal position for accessing the epidural space 60 because the orientation of the epidural space accessing device 10 is such that advancement of the epidural space accessing device 10 along trajectory 94 will eventually result in the device 10 hitting the spinal process 78. In some embodiments, the lack of desirable position for the epidural space accessing device 10 may be determined by the doctor viewing the visual indicators in the display 404. Alternatively or additionally, the processing unit may not activate an indicator (e.g., LED indicator, speaker, etc.) to emit a signal to indicate that a desirable entry trajectory has been reached, or the processing unit may cause the indicator to emit a signal to let the doctor know that a desired entry trajectory has not been reached.

Upon knowing that a desired entry trajectory still has not been reached, the doctor continuously repositions the epidural space accessing device 10, e.g., by translating, tilting, or both translating and tilting, the epidural space accessing device 10 using the handle 108. When the epidural space accessing device 10 is positioned like that shown in FIG. 18B, the trajectory 94 (as defined by the entry point 90b and the entry angle 92 relative to the patient's skin 80) will go through the interspinous ligament 84 between the spinal processes 78 to reach the ligamentum flavum 66.

From the visual indicators presented at the display 404, the doctor may determine that the position of the epidural space accessing device 10 is desirable. Alternatively, the processing unit in the user interface 402 may automatically determine for the doctor that a desired entry trajectory has been obtained. In some embodiments, the processing unit may activate an indicator (e.g., a LED indicator, or a speaker) to emit a signal to indicate to the doctor that a desired entry trajectory has been reached. Alternatively, the processor unit may stop the indicator from emitting a signal that indicates that a desired entry trajectory has not been reached.

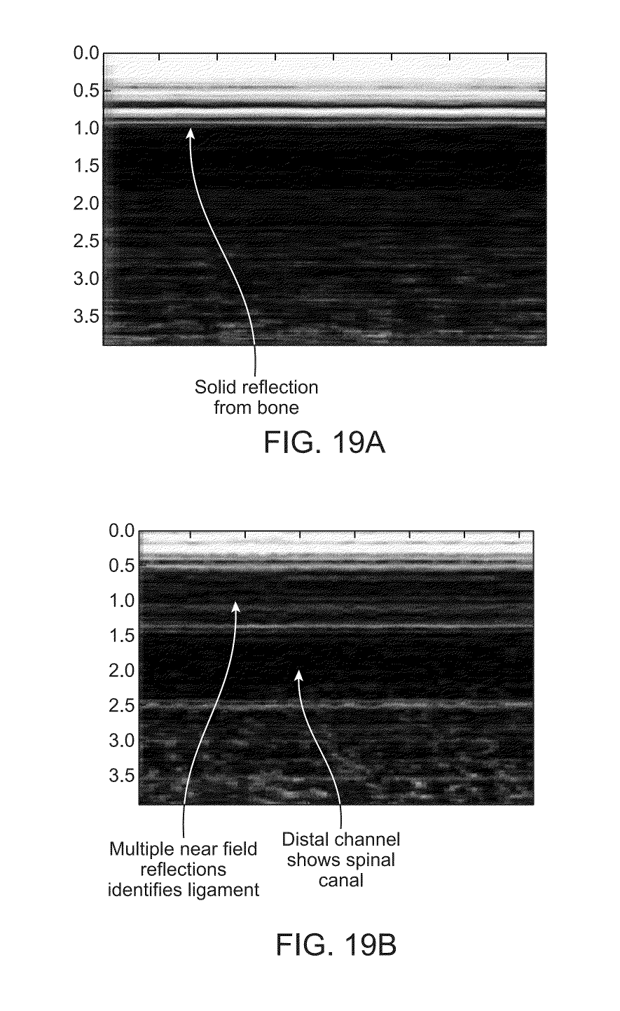

In some embodiments, the processing unit may compare the pattern of the lines in the display 404 with pre-determined templates of different patterns to see if a match can be found. For example, as shown in FIG. 19A, the pattern of lines that includes a thick isolated band may indicate that the acoustic reflection is from bone. Accordingly, in this example, the device is pointing towards bone, and the display indicating that the orientation of the device relative to the patient is not desirable. On the other hand, as shown in FIG. 19B, the pattern of lines may include multiple lines representing near field reflections with a distal channel showing spinal canal, indicating that the device is pointing away from bone, e.g., towards a ligament. Accordingly, in this example, the device is at a desired orientation relative to the patient.

In some embodiments, images like that shown in FIG. 19B may be stored as templates for comparison with real time images to see if a match is found. If a match is found, the processing unit may then automatically determine that a desired entry trajectory has been reached. Also, images like that shown in FIG. 19A may be stored as templates for comparison with real time images to see if a match is found. If a match is found, the processing unit may determine that the trajectory is pointing at bone, which is an undesirable trajectory. In other embodiments, instead of the processing unit determining whether the device is at a desired orientation relative to the patient, the real time images (like those shown in the examples of FIGS. 19A-19B) may be displayed on a screen (e.g., display 404) for allowing a user of the device to decide whether the device is at a desired orientation relative to the patient.

Alternatively or additionally, the processing unit may determine that a desired entry trajectory has been reached using timing information (e.g., time it takes for an acoustic signal to reflect back to the transducer component 120) and/or amplitude information (e.g., amplitude of reflected signal). In the configuration shown in FIG. 18B, the time it takes for an acoustic signal to reflect back from a bony structure to the transducer component 120 is relatively longer (longer than the configurations shown in FIG. 18A), and the reflected acoustic signal strength is relatively low (compared to the configurations shown in FIG. 18A).

Upon knowing, through the “successful” signal (or lack of “unsuccessful” signal) provided by the epidural space accessing device 10, or through visual examination of the indicators presented in the display 404, that the desired entry trajectory for accessing the epidural space 60 has been achieved, the doctor then advances the epidural space accessing device 10 along the trajectory 94 to puncture the skin 80, then the tissue 82, and then the interspinous ligament 84 (FIG. 18C). As shown in the illustrated embodiments, because the desired entry trajectory 94 has been established using the epidural space accessing device 10 before the epidural space accessing device 10 punctures the patient's skin 80 (or after the puncturing of the skin 80, but before the distal tip of the device passes through the tissue 82), the epidural space accessing device 10 does not hit any bony structure as the epidural space accessing device 10 is advanced towards the epidural space 60.

In some embodiments, after the device 10 has penetrated the patient's skin 80, the transducer component 120 continues to emit acoustic signals and receive reflected acoustic signals. Accordingly, the doctor may continue to view the display 404 to determine whether the epidural space accessing device 10 is being advanced along a desired path. If the graphics in the display 404 indicate that the epidural space accessing device 10 is being moved along an undesirable path, the doctor may then adjust the trajectory of the epidural space accessing 10. In other embodiments, the processing unit in the user interface 402 may process the reflected acoustic signals while the epidural space accessing device 10 is being advanced, and may determine whether the epidural space accessing device 10 is being desirably advanced through the tissue 82 and the interspinous ligament 84. In some cases, if the processing unit determines that the epidural space accessing device 10 is too close to the spinal process 78, the processing unit then generates a signal (e.g., a visual signal, an audio signal, or both) to inform the doctor that the trajectory of the epidural space accessing device 10 needs to be adjusted. In some embodiments, the signal may also inform the user how to adjust the device 10 (e.g., by informing the user the direction of adjustment).

As shown in FIG. 18D, the doctor continues to advance the epidural space accessing device 10 until the distal tip 105 has reached the epidural space 60. Various techniques may be employed to determine whether the distal tip 105 has reached the epidural space 60. In some embodiments, the proximal end of the handle 108 is coupled to a fluid source (e.g., a syringe). As the doctor is advancing the device 10, the doctor uses the syringe to apply fluid delivery pressure through the lumen 107 of the shaft 102. As the distal end 104 goes through the skin 80, tissue 82, the interspinous ligament 84, and ligamentum flavum 66, the distal opening at the distal end 104 is blocked by these tissues. Therefore, the doctor will sense high pressure at the syringe as the doctor advances the distal end 104 through these tissues. As soon as the distal tip 105 has gone past the ligamentum flavum 66, the distal tip 105 is in the epidural space 60, and the opening 109 at the distal end 104 is no longer blocked by tissue. As such, the doctor will sense that the pressure being applied at the syringe has dropped, thereby knowing that the distal tip 105 has reached the epidural space 60.

Once the distal tip 105 is placed in the epidural space 60, the doctor then uncouples the syringe from the handle 108, and inserts a catheter (not shown) through the handle into the lumen 107 of the shaft 102. The catheter is advanced until it exits through the distal opening 109 at the distal end 104 of the shaft 102, and is desirably placed in the epidural space 60. The catheter may then be used to deliver fluid, such as saline, pain medication, or other substance into the epidural space 60. Alternatively, the shaft 102 itself may be used to deliver fluid into the epidural space 60. For example, the handle 108 may be directly coupled to a fluid source, such as a source of saline, pain medication, or any other drugs, and the lumen 107 of the shaft 102 is then directly used to deliver such fluid into the epidural space 60. In other embodiments, the epidural space accessing device 10 may function as an introducer sheath that allows other devices, such as a tissue cutter, to be delivered to the epidural space 60 therethrough.

Also, in other embodiments, the distal tip 105 of the shaft 102 may not need to penetrate through the ligamentum flavum 66. In such cases, when the distal tip 105 reaches the ligamentum flavum 66, another device may be placed through the lumen 107 of the shaft 102, and may be deployed out of the distal opening 109 at the distal end 104 of the shaft 102 for penetrating the ligamentum flavum 66. FIGS. 18E-18G illustrate the tissue-cutting device 200 in FIG. 4 that may be used to penetrate through the ligamentum flavum 66. In some embodiments, the tissue-cutting device 200 may be considered to be a part of the epidural space accessing device 10. In the illustrated embodiments, the device 400 is removed from the lumen 107 of the shaft 102, and the tissue-cutting device 200 is inserted into the lumen 107 (FIG. 18E). Alternatively, if the shaft 102 has two lumens, then the device 400 may remain in a first lumen, while the tissue-cutting device 200 is inserted into a second lumen in the shaft 102. Such configuration may allow the positioning of the epidural space accessing device 10 be continued monitored while the tissue-cutting device 200 is being used.

As shown in FIG. 18F, after the distal end 104 of the shaft 102 is placed next to the ligamentum flavum 66, and after the tissue-cutting device 200 is then inserted into the lumen 107 through the handle 108. The tissue-cutting portion 210 is then deployed out of the distal opening at the distal end 104 of the epidural space accessing device 10, and is used to engage the ligamentum flavum 66. In particular, the doctor may rotate the handle 208 about the longitudinal axis 220 to screw the tissue-cutting portion 210 into the ligamentum flavum 66. The doctor continues to advance the tissue-cutting portion 210 by screwing action until the opening 214 at the tissue-cutting portion 210 has passed through the ligamentum flavum 66 into the epidural space 60 (FIG. 18G). Using the tissue-cutting portion 210 with the screw threads 212 to penetrate the ligamentum flavum 66 is advantageous because it allows the doctor to slowly and controllably advance the distal opening 214 of the tissue-cutting device 200 into the epidural space 60 in a safe manner. In some embodiments, while the doctor is advancing the tissue-cutting portion 210 through the ligamentum flavum 66, the proximal end of the handle 208 may be coupled to a fluid source (e.g., a syringe) 600, and the doctor may operate the syringe to apply fluid delivery pressure within the lumen 218, as similarly discussed. Before the distal opening 214 exits pass the ligamentum flavum 66, the doctor will sense high pressure while operating the syringe. However, as soon as the distal opening 214 exits pass the ligamentum flavum 66 into the epidural space 60, the doctor will sense that the pressure associated with using the syringe has dropped, thereby knowing that the distal opening 214 has reached the epidural space 60.

Next, a catheter may be inserted into the lumen 218 of the tissue-cutting device 200, and exits through the opening 214 into the epidural space 60. The catheter is then used to deliver fluid, such as saline, pain medication, or any other drugs, into the epidural space 60. Alternatively, the tissue-cutting device 200 itself may be used to deliver fluid, such as saline, pain medication, or any other drugs, into the epidural space 60.

In further embodiments, the shaft 102 of the epidural space accessing device 10 itself may include the tissue-cutting portion 210 with the screw threads 212 and the distal opening 214. Such configuration obviates the need to place a separate tissue-cutting device into the lumen 107 of the shaft 102. During use, after the doctor has placed the distal end 104 of the shaft 102 next to the ligamentum flavum 66, the doctor then rotates the shaft 102 about its longitudinal axis to screw the tissue-cutting portion 210 into the ligamentum flavum 66. The doctor continues to advance the tissue-cutting portion 210 by screwing action until the opening 214 at the tissue-cutting portion 210 has passed through the ligamentum flavum 66 into the epidural space 60. Next, the device 400 may be removed from the lumen 107 of the shaft 102, and a catheter may be inserted into the lumen 107 of the shaft 102, and exits through the opening 214 into the epidural space 60. The catheter is then used to deliver fluid, such as saline, pain medication, or any other drugs, into the epidural space 60. Alternatively, the lumen 107 of the shaft 102 itself may be used to deliver fluid, such as saline, pain medication, or any other drugs, into the epidural space 60. In further embodiments, the shaft 102 may include a first lumen for housing the device 400, and a second lumen for housing the fluid delivery catheter (or for directly delivering fluid). In some embodiments, while the doctor is advancing the tissue-cutting portion 210 through the ligamentum flavum 66, the proximal end of the handle 108 may be coupled to a fluid source (e.g., a syringe), and the doctor may operate the syringe to apply fluid delivery pressure within the lumen 107 (or another lumen), as similarly discussed. Before the distal opening 214 exits pass the ligamentum flavum 66, the doctor will sense high pressure while operating the syringe. However, as soon as the distal opening 214 exits pass the ligamentum flavum 66 into the epidural space 60, the doctor will sense that the pressure associated with using the syringe has dropped, thereby knowing that the distal opening 214 of the tissue-cutting portion 210 has reached the epidural space 60.

It should be noted that the configuration of the device 10 is not limited to the examples described herein, and that the device 10 may have other configurations in other embodiments. For example, in other embodiments, the device 10 may not have an elongated body. Instead, the device 10 may be a hand-held device having a housing with block-like configuration, or other configurations. Also, as illustrated in the above embodiments, providing the handle 108 for the device 10 is advantageous in that it allows the doctor to selectively position the device 10 at different positions relative to the patient. This in turn allows the doctor to obtain information about the internal bodily structure from different device's 10 positions, thereby enabling the doctor to find the desired entry point, desired entry angle, or both, for accessing the epidural space. However, in other embodiments, the device 10 may not include the handle 108, and the device 10 may be configured to be fixedly secured to the patient during use. For example, in some embodiments, the device 10 may be fixedly attached to the patient skin using an adhesive, a strap, a belt, or other known devices. In such cases, the device 10 may include a transmitter for transmitting a signal towards an internal part of the patient through the skin. The device 10 or another device (which may or may not be fixedly couple to the patient's skin) may have a sensor for sensing signal reflected from internal bodily structure. The user may then use the sensed signal to determine a desired entry point, a desired entry angle, or both, for accessing the epidural space.

As illustrated in the above embodiments, devices and methods described herein facilitate controlled entry into the epidural space by assisting a user to determine a desired entry point and/or entry angle before the patient's skin is punctured to access the epidural space 60. This is advantageous because it results in the device being inserted properly to thereby ensuring that patient's skin is punctured only once in some embodiments. In some embodiments, the devices and methods described herein also allows a user to continue monitoring the position of the epidural space accessing device while it is being advanced towards the epidural space 60. The effective passage that a device can navigate between the skin and the epidural space is narrow, bounded by vertebral bony structures, and is in proximity to important and delicate anatomical structures. As illustrated in the above embodiments, the devices and methods described herein are advantageous because they enable guidance of devices to the epidural space 60 while avoiding intersection with bony structures and delicate anatomical structures in the vicinity. Thus, the devices and methods described herein permit safe, effective, and efficient access to the epidural space for various medical procedures.

As discussed, in some embodiments, the user interface 402 of the device 10 may be implemented using a handheld communication device, such as an iPhone. FIGS. 20A-20B show an embodiment of the device 10 in which the user interface 402 is implemented using iPhone. The device 10 includes the iphone for implementing the user interface 402. As shown in the figure, the elongate device is communicatively coupled to the iPhone through a cable and an adaptor.

A person skilled in the art will appreciate the foregoing as only illustrative of the various embodiments, and that various modifications may be made to both the epidural space accessing devices and the methods presented without departing from the scope and spirit of the invention.

1. A device for use in a process that involves accessing an epidural space, comprising:
an elongated member having a distal end and a proximal end;
an ultrasound component located at the distal end of the elongated member;
a rigid tubular member having a tapered distal end, a proximal end, and a lumen extending between the distal end and the proximal end;
wherein the ultrasound component is configured to emit ultrasound in a distal direction, and to receive reflected ultrasound; and
wherein the elongated member is slidably disposed in the lumen of the tubular member.

2. The device of claim 1, further comprising a display for displaying information derived from the reflected ultrasound.

3. The device of claim 2, further comprising a handheld device, wherein the display is a part of the handheld device.

4. (canceled)

5. The device of claim 2, further comprising a computer, wherein the display is a part of the computer.

6. The device of claim 2, further comprising circuit for processing signals derived from the reflected ultrasound.

7. The device of claim 6, wherein the circuit comprises a processor.

8. The device of claim 2, wherein the information comprises a series of lines displayed in the display, each of the lines corresponding to a respective tissue depth.

9-12. (canceled)

13. The device of claim 2, wherein the information is configured to provide a sensory indication for assisting a user to identify a desired entry path to access an epidural space.

14. The device of claim 1, wherein the ultrasound component comprises a transducer body having an end surface, a circumferential surface, a first channel at the circumferential surface, and a second channel at the end surface.

15. The device of claim 14, wherein the ultrasound component further comprises a wire, wherein the wire has a first segment disposed in the first channel, and a second segment that is distal to the first segment, the second segment disposed in the second channel and is bent 90° relative to the first segment.

16-22. (canceled)

23. The device of claim 1, further comprising a tissue-cutting device configured for insertion into the tubular member.

24. The device of claim 23, wherein the tissue-cutting device comprises an elongated body and screw threads for engagement with a ligamentum flavum at a distal end of the elongated body.

25. (canceled)

26. A device for use in a process that involves accessing an epidural space, comprising:
an elongated member having a distal end and a proximal end;
an ultrasound component located at the distal end of the elongated member, wherein the ultrasound component is configured to receive reflected ultrasound, and generate an output based on the reflected ultrasound;
a rigid tubular member having a tapered distal end, a proximal end, and a lumen extending between the distal end and the proximal end, wherein the elongated member is slidably disposed in the lumen of the tubular member; and
a processing unit communicatively coupled to the ultrasound component, wherein the processing unit is configured to generate signals based on the output from the ultrasound component, the signals representing information for presentation in a display, wherein the information comprises a series of lines, each of the lines corresponding to a respective tissue depth.

27. The device of claim 26, further comprising the display for receiving the signals from the processing unit, and displaying the information based on the signals.

28. The device of claim 27, further comprising a handheld device, wherein the display is a part of the handheld device.

29. (canceled)

30. The device of claim 27, further comprising a computer, wherein the display is a part of the computer.

31. The device of claim 26, wherein the lines are dynamically changing based on a positioning of the ultrasound component.

32-33. (canceled)

34. The device of claim 26, wherein the information is configured to provide a sensory indication for assisting a user to identify a desired entry path to access an epidural space.

35. The device of claim 26, wherein the ultrasound component comprises a body having an end surface, a circumferential surface, a first channel at the circumferential surface, and a second channel at the end surface.

36. The device of claim 35, wherein the ultrasound component further comprises a wire, wherein the wire has a first segment disposed in the first channel, and a second segment that is distal to the first segment, the second segment disposed in the second channel and is bent 90° relative to the first segment.

37. The device of claim 26, further comprising a tissue-cutting device configured for insertion into the tubular member.

38. The device of claim 37, wherein the tissue-cutting device comprises an elongated body and screw threads for engagement with a ligamentum flavum at a distal end of the elongated body.

39. (canceled)

40. The device of claim 26, wherein the processor is configured to generate an indicator signal when the ultrasound component is at a desired entry point to access the epidural space.

41. The device of claim 26, wherein the processor is configured to generate an indicator signal when the ultrasound component is not at a desired entry point to access the epidural space.

42-53. (canceled)
